SEQUENCE LISTING (58 sequences)

SEQ ID NO: 1 - DNA, 21 bp, Homo sapiens
  source 1..21: mol_type = other DNA
cgcttgtaaataccaggattt

SEQ ID NO: 2 - DNA, 21 bp, Homo sapiens
  source 1..21: mol_type = other DNA
cggcatgaacatgagcggcat

SEQ ID NO: 3 - DNA, 21 bp, Mus musculus
  source 1..21: mol_type = other DNA
cggcatgaatatgagtggcat

SEQ ID NO: 4 - DNA, 21 bp, Mus musculus
  source 1..21: mol_type = other DNA
gttctcagtgtctgacatctt

SEQ ID NO: 5 - DNA, 20 bp, Mus musculus
  source 1..20: mol_type = genomic DNA
catgtcgatgagtccaaagc

SEQ ID NO: 6 - DNA, 20 bp, Mus musculus
  source 1..20: mol_type = genomic DNA
ctccatgcccactttcttgt

SEQ ID NO: 7 - DNA, 20 bp, Mus musculus
  source 1..20: mol_type = genomic DNA
tgcacccaaaccgaagtcat

SEQ ID NO: 8 - DNA, 21 bp, Mus musculus
  source 1..21: mol_type = genomic DNA
acttggggacaccttttagca

SEQ ID NO: 9 - DNA, 22 bp, Mus musculus
  source 1..22: mol_type = genomic DNA
ctgccaagggttgacttcaaga

SEQ ID NO: 10 - DNA, 21 bp, Mus musculus
  source 1..21: mol_type = genomic DNA
gcttcagggtcaaggcaaact

SEQ ID NO: 11 - DNA, 21 bp, Mus musculus
  source 1..21: mol_type = genomic DNA
ccagacagaagtcatagccac

SEQ ID NO: 12 - DNA, 21 bp, Mus musculus
  source 1..21: mol_type = genomic DNA
cttcatcatggtgaggggctt

SEQ ID NO: 13 - DNA, 20 bp, Mus musculus
  source 1..20: mol_type = genomic DNA
tgccctacggtggaagtcat

SEQ ID NO: 14 - DNA, 18 bp, Mus musculus
  source 1..18: mol_type = genomic DNA
gcgagtgcattccgctta

SEQ ID NO: 15 - DNA, 20 bp, Mus musculus
  source 1..20: mol_type = genomic DNA
ggaggaacctgccaagtatg

SEQ ID NO: 16 - DNA, 18 bp, Mus musculus
  source 1..18: mol_type = genomic DNA
tgggagttgctgttgaag

SEQ ID NO: 17 - DNA, 20 bp, Mus musculus
  source 1..20: mol_type = genomic DNA
aagaaccgacaaggacacgg

SEQ ID NO: 18 - DNA, 20 bp, Mus musculus
  source 1..20: mol_type = genomic DNA
tctgatggtcgtaggggctt

SEQ ID NO: 19 - DNA, 20 bp, Mus musculus
  source 1..20: mol_type = genomic DNA
atgtcctgggcagagtgaga

SEQ ID NO: 20 - DNA, 20 bp, Mus musculus
  source 1..20: mol_type = genomic DNA
tggagctttagatgccgctt

SEQ ID NO: 21 - DNA, 20 bp, Mus musculus
  source 1..20: mol_type = genomic DNA
ttctctggcacgtcttgacc

SEQ ID NO: 22 - DNA, 20 bp, Mus musculus
  source 1..20: mol_type = genomic DNA
gcttggaaacgtccacatcg

SEQ ID NO: 23 - DNA, 20 bp, Mus musculus
  source 1..20: mol_type = genomic DNA
caaccctgggtatgacaccg

SEQ ID NO: 24 - DNA, 20 bp, Mus musculus
  source 1..20: mol_type = genomic DNA
ccgcctaaagccatgttcct

SEQ ID NO: 25 - DNA, 19 bp, Homo sapiens
  source 1..19: mol_type = genomic DNA
agcacacgactccgttctc

SEQ ID NO: 26 - DNA, 22 bp, Homo sapiens
  source 1..22: mol_type = genomic DNA
gcccactttcttgtagctttcc

SEQ ID NO: 27 - DNA, 20 bp, Homo sapiens
  source 1..20: mol_type = genomic DNA
caccccaagaacatccaaag

SEQ ID NO: 28 - DNA, 20 bp, Homo sapiens
  source 1..20: mol_type = genomic DNA
taactatgggggatgcagga

SEQ ID NO: 29 - DNA, 22 bp, Homo sapiens
  source 1..22: mol_type = genomic DNA
cacctcaagaacatccaaagtg

SEQ ID NO: 30 - DNA, 20 bp, Homo sapiens
  source 1..20: mol_type = genomic DNA
gattttcttaaccatgggcg

SEQ ID NO: 31 - DNA, 20 bp, Homo sapiens
  source 1..20: mol_type = genomic DNA
gagcgtccgtggtcactgaa

SEQ ID NO: 32 - DNA, 20 bp, Homo sapiens
  source 1..20: mol_type = genomic DNA
ccgggggaccttacattcac

SEQ ID NO: 33 - DNA, 20 bp, Homo sapiens
  source 1..20: mol_type = genomic DNA
accacgcaaggagttcatcc

SEQ ID NO: 34 - DNA, 20 bp, Homo sapiens
  source 1..20: mol_type = genomic DNA
ggggcttctggatcaagaca

SEQ ID NO: 35 - DNA, 22 bp, Homo sapiens
  source 1..22: mol_type = genomic DNA
gcagggtctaaaagctggtgtt

SEQ ID NO: 36 - DNA, 22 bp, Homo sapiens
  source 1..22: mol_type = genomic DNA
tccctatgcatctcacccatct

SEQ ID NO: 37 - DNA, 21 bp, Homo sapiens
  source 1..21: mol_type = genomic DNA
atttttccctcgacacccgat

SEQ ID NO: 38 - DNA, 19 bp, Homo sapiens
  source 1..19: mol_type = genomic DNA
tcccaggcgtagaccaaga

SEQ ID NO: 39 - DNA, 20 bp, Homo sapiens
  source 1..20: mol_type = genomic DNA
ggcgaggagagcaggatttc

SEQ ID NO: 40 - DNA, 21 bp, Homo sapiens
  source 1..21: mol_type = genomic DNA
agtgggtatcaaccagaggga

SEQ ID NO: 41 - DNA, 20 bp, Homo sapiens
  source 1..20: mol_type = genomic DNA
acccccaccagcctactgat

SEQ ID NO: 42 - DNA, 20 bp, Homo sapiens
  source 1..20: mol_type = genomic DNA
acttgctcccaggcacagtg

SEQ ID NO: 43 - DNA, 18 bp, Homo sapiens
  source 1..18: mol_type = genomic DNA
agcagtgaaagagaaggg

SEQ ID NO: 44 - DNA, 18 bp, Homo sapiens
  source 1..18: mol_type = genomic DNA
ggtcctcttcaggtgcct

SEQ ID NO: 45 - DNA, 23 bp, Homo sapiens
  source 1..23: mol_type = genomic DNA
cctctgtagatggtccagaagaa

SEQ ID NO: 46 - DNA, 23 bp, Homo sapiens
  source 1..23: mol_type = genomic DNA
ctgtaccattgttaattgcggtc

SEQ ID NO: 47 - DNA, 22 bp, Homo sapiens
  source 1..22: mol_type = genomic DNA
ccagccccaaccatgcataaaa

SEQ ID NO: 48 - DNA, 21 bp, Homo sapiens
  source 1..21: mol_type = genomic DNA
ctgtgagaggagcggaagagc

SEQ ID NO: 49 - DNA, 22 bp, Homo sapiens
  source 1..22: mol_type = genomic DNA
actctacctgcacactgtccta

SEQ ID NO: 50 - DNA, 24 bp, Homo sapiens
  source 1..24: mol_type = genomic DNA
atgactggagaacatttgaaaaca

SEQ ID NO: 51 - DNA, 20 bp, Homo sapiens
  source 1..20: mol_type = genomic DNA
attcccggagctccagatcg

SEQ ID NO: 52 - DNA, 20 bp, Homo sapiens
  source 1..20: mol_type = genomic DNA
aagacagtcagacccggacg

SEQ ID NO: 53 - DNA, 23 bp, Homo sapiens
  source 1..23: mol_type = genomic DNA
tgctacaacaatgttcaaagtca

SEQ ID NO: 54 - DNA, 25 bp, Homo sapiens
  source 1..25: mol_type = genomic DNA
gcattgggattttatagattctgga

SEQ ID NO: 55 - DNA, 19 bp, Homo sapiens
  source 1..19: mol_type = genomic DNA
catagtggtcaagagagcg

SEQ ID NO: 56 - DNA, 20 bp, Homo sapiens
  source 1..20: mol_type = genomic DNA
ggggagagatgagtgtagat

SEQ ID NO: 57 - DNA, 21 bp, Homo sapiens
  source 1..21: mol_type = genomic DNA
aaacataacttagtgacaagc

SEQ ID NO: 58 - DNA, 21 bp, Homo sapiens
  source 1..21: mol_type = genomic DNA
ctctgtacgatttaagtaaca

(19) United States
(12) Patent Application Publication
(10) Pub. No.: US 2026/0266822 A1
(43) Pub. Date: Sep. 10, 2026

(54) BIOMARKERS FOR LUNG ADENOCARCINOMA (LUAD), AND METHOD FOR TREATING LUAD

(71) Applicant: TAIPEI VETERANS GENERAL HOSPITAL, Taipei City (TW)

(72) Inventors: Mong-Lien WANG, Taipei City (TW); Shih-Hwa CHIOU, Taipei City (TW); Chih-Ying CHEN, Taipei City (TW); Ping-Hsing TSAI, Taipei City (TW); Lo-Jei CHING, Taipei City (TW); Ming-Long TSAI, Taipei City (TW); Yuh-Min CHEN, Taipei City (TW); Yung-Hung LUO, Taipei City (TW); Po-Kuei HSU, Taipei City (TW)

(73) Assignee: TAIPEI VETERANS GENERAL HOSPITAL, Taipei City (TW)

(21) Appl. No.: 19/075,628

(22) Filed: Mar. 10, 2025

(51) Int. Cl. G01N 33/574; A61K 31/17; A61P 35/00

(52) U.S. Cl. CPC G01N 33/5752; A61K 31/17; A61P 35/00; G01N 2333/4703

(57) ABSTRACT

The present invention provides a method for predicting clinical outcomes of a lung cancer patient, such as LUAD, particularly LUAD with low expression of NKX2-1. Also provided includes a method for treating lung adenocarcinoma (LUAD) in a patient, comprising administering to said patient an inhibitor of CXCR2 at a therapeutically effective amount. The present invention provides a strategy of treatment to suppress tumorigenesis and improve the survival outcome of LUAD patients with NKX2-1-low malignant tumors through targeting the NKX2-1/CXC chemokine signaling.

### FIELD OF THE INVENTION

The present invention relates to biomarkers for lung adenocarcinoma (LUAD) and a method for treating LUAD.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 10, 2025, is named “CSH0023US Sequence Listing” and is 51,684 bytes in size. The sequence listing contained in this .XML file is part of the specification and is hereby incorporated by reference herein in its entirety.

### BACKGROUND OF THE INVENTION

Lung cancer is the most prevalent and aggressive cancer worldwide, with non-small-cell lung carcinoma (NSCLC) comprising approximately 85% of lung cancer cases. In turn, NSCLC can be classified into lung adenocarcinoma (LUAD), squamous cell carcinoma (SCC), and large cell lung carcinoma (LCLC) [1]. LUAD is a common NSCLC subtype associated with the highest mortality, increased recurrence rate, and short relapse-free survival [2]. Despite the advancements in lung cancer treatment, the prognosis of LUAD patients remains poor due to the challenges posed by metastasis and drug resistance [3]. High-grade LUAD is characterized by elevated tumor plasticity manifested in frequent cellular and molecular transitions, such as epithelial-mesenchymal transition (EMT), a process critical for metastasis [4]. Previous studies have shown that the alteration of the tumor microenvironment (TME) affects the infiltration of immune cells with concurrent LUAD tumor progression [5]. However, the detailed mechanisms governing the LUAD-associated immune microenvironment, which involves a complex interplay of immune cells, cytokines, and paracrine-driven molecules interacting with the tumor, remain unclear.

The immune microenvironment comprises of an intricate network of immune cells, cytokines, and various molecules interacting with cancer cells within the TME [6]. Among them are neutrophils, a type of innate immune cells that play a vital role in responding to infection and inflammation [7]. Neutrophils are abundant in the bloodstream, comprising approximately 70% of all human white blood cells and 10-20% in mice [8]. The activation of CXCR2 is a critical step in triggering the migration of neutrophils from the bone marrow [9], whereas CXCR4 regulates the retention of neutrophils in the bone marrow [10]. In solid tumors, neutrophils can exhibit anti-tumorigenic activity involving cytotoxicity against tumor cells and promote tumor growth, angiogenesis, metastasis, and immune evasion [8, 11]. The infiltration of tumors with neutrophils is mediated through the chemotactic effects of CXC chemokines by binding to their receptor, CXCR2 [12]. For instance, CXCL8 is a common ligand of CXCR2 that is mainly secreted by different types of tumors and controls neutrophil recruitment [13]. Additionally, the activation of CXCLs/CXCR2 signaling promotes tumor angiogenesis, metastasis, chemoresistance, and tumor progression [14, 15].

Cell plasticity facilitates the transition between cell lineages and the emergence of drug resistance in specific cancer subtypes, contributing to increased tumor aggressiveness [16]. These transitions are characterized by massive changes in gene expression programs regulated by master regulator transcription factors (TFs). Among them is NK2 homeobox 1 (NKX2-1), which is predominantly expressed during lung and thyroid development and serves as a lineage-specific TF that determines alveolar cell identity [18]. In lung cancer, NKX2-1 regulates the identity of LUAD by enforcing differentiation programs; meanwhile, NKX2-1 downregulation confers worse disease outcomes in poorly differentiated tumors [19]. On the contrary, transdifferentiation from adenomatous to more aggressive squamous histological type is associated with elevated transcriptional activity of SOX2, a well-known TF in squamous cell carcinoma [20]. SOX2 is known to change squamous cell carcinoma phenotype to a club and alveolar type 2 (AT2) cells [20]. Furthermore, it enhances the generation of neural progenitor cells in lung epithelial cells [21]. The balance between SOX2 and NKX2-1 plays a critical role in determining the shifts in cellular lineage that modulate lung cancer progression due to the property of lineage-specific TFs to govern differentiation status that is typically associated with the degree of tumor malignancy [18].

Recently, it has been demonstrated that neutrophil plasticity and heterogeneity underlie adverse events that may result in the discontinuation of immunotherapy [8]. Previous reports have shown that SOX2 is a critical TF that recruits neutrophils into the TME by regulating CXCL3 and CXCL5 expression, thus facilitating cancer progression [16]. However, the implication of NKX2-1 in attracting immune cells to the TME and mediating the progression of LUAD remains unclear.

It is desirable to find one or more biomarkers for LUAD and develop a method for diagnosing and treating LUAD based on the biomarkers.

### BRIEF SUMMARY OF THE INVENTION

It is unexpectedly found in the present invention that the activation of CXCLs/CXCR2 signaling provides a cell-cell communication between NKX2-1-low tumors and neutrophils, and low expression of NKX2-1 correlates with high neutrophil infiltration, which predicts poor clinical outcomes in lung adenocarcinoma (LUAD) patients.

In one aspect, the present invention provides a method for predicting clinical outcomes of a lung cancer patient, comprising providing a cancer cell sample of the patient, and detecting the expression level of NKX2-1 in the sample, wherein the clinical outcome is predicted as poor if the expression of NKX2-1 is low.

In one embodiment of the present invention, the lung cancer is lung adenocarcinoma (LUAD).

In another aspect, the present invention provides a method for treating lung adenocarcinoma (LUAD) in a patient, comprising administering to said patient an inhibitor of CXCR2 at a therapeutically effective amount.

In one yet aspect, the present invention provides a use of an inhibitor of CXCR2 in manufacturing a medicament for treating lung adenocarcinoma (LUAD).

In one example, the LUAD is a cancer with low expression of NKX2-1.

In one example, the inhibitor of CXCR2 is CXCL1, CXCL2, or CXCL5.

In one example, the inhibitor of CXCR2 is a CXCR2 antagonist, which comprises SB225002, MK-7123, Cpd 19, or AZD5069.

In the present invention, the single-cell and visium in situ capturing NGS analyses revealed a strong cell-cell communication between NKX2-1-low tumors and neutrophils through the activation of CXCLs/CXCR2 signaling. The inhibition of CXCR2 chemokine receptor with specific inhibitor, such as SB225002, decreased the infiltration of tumor-promoting neutrophils, resulting in reduced tumor growth in NKX2-1-low tumors.

According to the invention, NKX2-1 plays a role as a bona fide modulator of the immune tumor microenvironment in NKX2-1-low LUAD, whose loss induces the expression/secretion of CXC chemokines leading to increased recruitment and infiltration of tumor-promoting neutrophils. These neutrophils exhibit cancer-promoting properties with strong cell-cell communication within tumor tissue. This creates a pro-oncogenic tumor microenvironment (TME), which ultimately drives increased tumor progression. Furthermore, the present invention provides that targeting the NKX2-1/CXC chemokine signaling may represent a strategy of treatment to suppress tumorigenesis and improve the survival outcome of LUAD patients with NKX2-1-low malignant tumors.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIGS. 1A-1C show the low expression of NKX2-1 is associated with aggressive LUAD. FIG. 1A shows a representative image of NKX2-1 IHC staining was performed on tumor tissue microarrays of different grades of LUAD. FIG. 1B and FIG. 1C show the histoscore (H-score) quantification of NKX2-1 IHC staining performed on tissue microarray of different LUAD grades (FIG. 1B) and stages (FIG. 1C). The data are presented as means+SD error bars, N=3, **p<0.01, ***p<0.001, ****p<0.0001, ns—not significant (Student's t-test).

FIGS. 2A-2B show the Kaplan-Meier curves. FIG. 2A shows the overall survival; and FIG. 2B shows disease-free survival of NKX2-1 LUAD patients from the TCGA dataset. p-values were calculated by the log-rank test.

FIG. 3 shows qRT-PCR analysis providing the expression levels of NKX2-1 in LL2 cells expressing luciferase reporter and eGFP transfected with NKX2-1-targeting shRNAs (shNKX2-1/LL2 #1 and shNKX2-1/LL2 #2). Mean fold changes (N=3) relative to shRNA control (shCtrl/LL2) are shown with SD error bars, *p<0.05, **p<0.01, ***p<0.001, ****p<0.0001, ns—not significant (Student's t-test).

FIGS. 4A-4C show the low expression of NKX2-1 is associated with aggressive LUAD. FIG. 4A provides the schematic representation showing the design of experiments performed on both orthotopic and tail vein injected mouse models by using shNKX2-1/LL2 and shCtrl/LL2 cells.

FIG. 4B provides photographs (top panels) and GFP fluorescence images (bottom panels) of tumors derived from orthotopically injected shCtrl/LL2 (left panel) and shNKX2-1/LL2 cells (middle and right panels). FIG. 4C shows the hematoxylin-eosin staining and IHC staining of NKX2-1 were performed on the tumors derived from orthotopically injected shCtrl/LL2 and shNKX2-1/LL2 cells. Left panel: representative images. Right panel: H-score quantification. The data is presented as means±SD error bars, N=3, ****p<0.0001 (Student's t-test).

FIG. 5 shows the immunoblotting and qRT-PCR analysis of expression levels of NKX2-1 and EMT markers in LUAD samples.

FIG. 6 shows the IHC staining of EMT markers (E-cadherin and Fibronectin) performed on excised mouse tumor tissues derived from orthotopically injected shCtrl/LL2 and shNKX2-1/LL2 cells. Left panel: representative images. Right panel: H-score quantification. The data are presented as means±SD error bars, ****p<0.0001 (Student's t-test).

FIGS. 7A-7B show bioluminescent signal visualization (FIG. 7A) and photographs (FIG. 7B) of tumors derived from shCtrl/LL2 and shNKX2-1/LL2 cells injected via the tail vein.

FIG. 8 shows scatter plots providing the correlation between NKX2-1 expression and the indicated immune cell infiltrate levels in LUAD from TIMER dataset analysis. The gene expression levels against tumor purity are displayed first because genes highly expressed in the tumor microenvironment are expected to exhibit negative associations with tumor purity and vice versa.

FIGS. 9A-9B show that NKX2-1 expression negatively correlated with neutrophil infiltration. FIG. 9A show the representative images of IHC staining performed on tumor tissue microarrays of NKX2-1-positive and negative LUAD tissues showing the expression of neutrophil markers (ITGAM, CEACAM8, ELANE, and CXCR2). FIG. 9B provides the H-score quantification of the expression levels of the selected neutrophil markers (ITGAM, CEACAM8, ELANE, and CXCR2) performed on tumor tissue microarrays of NKX2-1-positive and negative LUAD tissues. Data are presented as means±SD error bars, ****p<0.0001 (Student's t-test).

FIG. 10 shows the TCGA dataset analysis providing the correlation between NKX2-1 and the selected neutrophil markers (ITGAM, CEACAM8, ELANE, and CXCR2) in LUAD.

FIG. 11 shows the relapse-free survival curve providing the survival of LUAD patients with high or low neutrophil infiltration from TCGA dataset analysis. p-values were calculated by the log-rank test.

FIG. 12 shows the IHC staining of the indicated neutrophil markers in the cross sections of shCtrl/LL2 and shNKX2-1/LL2-derived tumors from mouse orthotopic model. Left panel: representative images. Right panel: H-score quantification. The data are presented as means±SD error bar, N=3, ***p<0.001, ****p<0.0001 (Student's t-test).

FIG. 13 shows a schematic illustration providing that NKX2-1-low LUAD tumors are characterized by high infiltration of neutrophils.

FIGS. 14A-14I show that NKX2-1-low tumors promote neutrophil infiltration via CXC chemokines. FIG. 14A shows the diagram demonstrating the experimental procedure for scRNA-seq and Visium in situ capturing. FIG. 14B shows UMAP analysis from scRNA-seq showing the distinct cell populations across two samples (shCtrl/LL2 and shNKX2-1/LL2-derived tumors).

FIGS. 14C and 14D show iTALK analysis of scRNA-seq validating the crosstalk between tumor cells and immune cells (FIG. 14C) and cell-to-cell communication (FIG. 14D) between cell types and the involved genes. FIG. 14E shows gene ontology biological process (GO-BP) enrichment analysis of differentially expressed genes in shNKX2-1/LL2 tumors compared to shCtrl/LL2 tumors identified by scRNA-seq. FIG. 14F shows UMAP analysis from scRNA-seq showing myeloid leukocyte migration in shCtrl/LL2 and shNKX2-1/LL2 tumors. FIG. 14G shows the hematoxylin-eosin staining (H&E) section annotations in shCtrl/LL2 and shNKX2-1/LL2 tumors (top panel) and representative image from Visium in situ capturing showing the activation of intracellular signal transduction and myeloid leukocyte activation. FIG. 14H and FIG. 14I show flow cytometry identification of Ly-6G+CD11b⁺ cells from healthy lungs and metastasized tumor-bearing lungs (shCtrl/LL2 and shNKX2-1/LL2) (FIG. 14H). The quantification of Ly-6G+CD11b+ cells (FIG. 14I) is shown as means±SD error bars, **p<0.01 (Student's t-test).

FIGS. 15A-15B show immunoblotting and qRT-PCR analysis validating the expression of NKX2-1 in H1975 (FIG. 15A) and HCC827 (FIG. 15B) cells by transfected with NKX2-1-targeting shRNAs (shNKX2-1 #1 and shNKX2-1 #2). Top: immunoblotting, and bottom: qRT-PCR. Mean fold changes (N=3) relative to shCtrl are shown with SD error bars, *p<0.05, **p<0.01, ****p<0.0001 (Student's t-test).

FIGS. 16A-16B show Functional enrichment analysis. The enrichment of GO biological process (GO-BP) (FIG. 16A) and molecular function (GO-MF) (FIG. 16B) terms among the genes positively regulated by NKX2-1 knockdown.

FIG. 17 shows the hierarchical clustering heatmap providing the differential gene expression (left panel) and the expression of CXC chemokines (right panel) upon NKX2-1 knockdown.

FIGS. 18A-18B show the qPCR analysis providing the expression level of CXC chemokines in HCC827 (FIG. 18A) and H1975 (FIG. 18B) resulting from the NKX2-1 knockdown. The mean fold changes (N=3) relative to scrambled shRNA control (shCtrl) are shown with SD error bars, *p<0.05, **p<0.01, ***p<0.001 (Student's t-test).

FIGS. 19A-19B show the chemokine array analysis providing the secretion levels of the indicated chemokines following NKX2-1 knockdown in HCC827 (FIG. 19A) and H1975 cells (FIG. 19B). Left panel: representative dot plot. Right panel: densitometry quantification of dot blots. *p<0.05, **p<0.01, ***p<0.001, ns—not significant (Student's t-test).

FIG. 20 shows TCGA dataset analysis providing the correlation between NKX2-1 expression and CXCL1, CXCL2, CXCL3, and CXCL5 expression levels.

FIG. 21 shows the immunofluorescence staining providing the localization of NKX2-1 in the nucleus of HCC827 and H1975 cells.

FIGS. 22A-22B show that NKX2-1 suppresses the chromatin accessibility of CXC chemokine genes at the promoter regions. FIG. 22A shows Heatmap representation of the chromatin accessibility at multiple genes' transcription start sites (TSS) and 10 kb upstream and downstream regions. FIG. 22B shows the annotation for ATAC-seq peak localization showing the distribution of sequenced reads across the indicated functional genomic elements. The peaks located 1 Kb upstream and 100 bp downstream of the TSS were defined as promoter-TSS.

FIG. 23 shows the distribution of open chromatin areas across the chromosomes shown as the ratio of signals from shNKX2-1/H1975 vs shCtrl/H1975.

FIG. 24 shows the GO-BP analysis of genes with open (top panel) and closed chromatin structure (bottom panel) at the promoter regions resultant from NKX2-1 knockdown.

FIG. 25 shows Integrative Genomics Viewer (IGV) representation of ATAC-seq signals at the promoter regions of CXC chemokine genes in shCtrl/H1975 and shNKX2-1/H1975 samples. The red line denotes a given range across the two samples.

FIGS. 26A-26B show NKX2-1 suppresses the chromatin accessibility at the promoter regions of CXC chemokine genes. FIG. 26A shows ChIP-qPCR analysis showing the binding of NKX2-1 at the promoter region of CXCL1, CXCL2, and CXCL5 genes in shNKX2-1/H1975 cells and H1975/AZDR overexpressing NKX2-1. Mean fold changes (N=3) relative to input are shown with SD error bars *p<0.05, **p<0.01, ***p<0.001 (Student's t-test). FIG. 26B shows the immunoblotting and qRT-PCR analysis providing the expression levels of NKX2-1 in the indicated parental (H1975 and HCC827) and EGFR-TKI-resistant (H1975/AZDR and HCC827/GRC) LUAD cells. GAPDH-loading control. Mean fold changes (N=3) relative to parental cells are shown with SD error bars, **p<0.01, ***p<0.001, ****p<0.0001, ns—not significant (Student's t-test).

FIG. 27 shows the most enriched canonical NKX2-1 binding motifs across ATAC-seq reads.

FIG. 28 shows the schematic illustration providing the potential mechanisms of NKX2-1 regulating the expression of CXC chemokines by altering chromatin structure.

FIG. 29 shows the schematic illustration demonstrating the experimental procedure for chemotaxis assay using HL-60 and conditioned medium from LUAD cell lines.

FIG. 30 shows the chemotaxis assay providing the migratory capacity of HL-60 cells in response to the conditioned media (CM) derived from H1975 (left panel) and HCC827 (right panel) subjected to NKX2-1 knockdown. Mean numbers of migrated cells (N=3) are shown with SD error bars, *p<0.05, **p<0.01, ***p<0.001 (Student's t-test).

FIG. 31 shows the revigo scatter plot from RNA-seq providing enriched GO-BP terms among the genes upregulated in HL-60 cells cultured with the conditioned media derived from shNKX2-1/H1975 as compared to shCtrl/H1975 as shown by RNA-seq analysis.

FIG. 32 shows the hierarchical clustering heatmap from RNA-seq providing the differential gene expression (left panel) and neutrophil-related genes (right panel) in HL-60 cultured with the conditioned media derived from shCtrl/H1975 and shNKX2-1/H1975.

FIG. 33 shows qRT-PCR analysis providing the expression levels of pro-inflammatory genes and anti-inflammatory genes in HL-60 cultured with conditioned medium derived from H1975 and HCC827 cells subjected to NKX2-1 knockdown. The mean fold changes (N=3) relative to shCtrl are shown with SD error bars, bars **p<0.01, ***p<0.001, ****p<0.0001, ns—not significant (Student's t-test).

FIG. 34 shows GO-BP enrichment analysis of the genes upregulated in the tumor regions derived from shNKX2-1/LL2 cells compared to shCtrl/LL2 cells from Visium in situ capturing profiling.

FIG. 35 shows the GO-BP enrichment analysis of the upregulated genes in tumors derived from shNKX2-1/LL2 cells compared to shCtrl/LL2 from Visium in situ capturing profiling. Upregulated genes were defined by −log 2 fold-change of more than 1 were defined as infiltration-positive, while the regions with below-threshold expression were defined as infiltration-negative.

FIG. 36 shows the violin plots providing the expression levels of the most upregulated genes annotated by GO-BP terms “regulation of cell communication” and “regulation of cell population proliferation” in shNKX2-1 and shCtrl infiltrated neutrophils.

FIG. 37 shows the visium in situ capturing representative images providing the neutrophil-positive regions and overlapping regions of the expression of genes annotated by “regulation of cell communication” and “regulation of cell population proliferation” GO-BP terms.

FIGS. 38A-38B show that in vivo targeting of CXCLs/CXCR2 signaling with SB225002 reduces tumor growth and neutrophil infiltration. FIG. 38A shows the subcutaneous mouse model. Top panel-schematic representation of the experimental design, bottom panel-tumor growth curve for 18 days following subcutaneous injection. Administration of SB225002 and vehicle control in the shNKX2-1/LL2 tumor. Data are shown as means±SD error bars, N=4, ***p<0.001 (Student's t-test). FIG. 38B shows the orthotopic mouse model. Top panel-schematic representation of the experimental design, bottom panel-bioluminescent signal visualization (top), photographs and GFP fluorescence images of tumors (bottom) derived from shCtrl/LL2, shNKX2-1/LL2, and shNKX2-1/LL2 injected with or without (vehicle) SB225002 administration.

FIG. 39 shows the result of chemotaxis assay showing the migratory capacity of HL-60 cells in response to the conditioned media (CM) containing SB225002 derived from H1975 (left panel) and HCC827 (right panel) subjected to NKX2-1 knockdown. Mean numbers of migrated cells (N=3) are shown with SD error bars, **p<0.01, ***p<0.001, ****p<0.0001, ns—not significant (Student's t-test).

FIG. 40 shows the violin plot providing the neutrophil markers used to distinguish the neutrophil population from other cell types in scRNA-seq analysis.

FIGS. 41A-41B show the UMAP analysis from scRNA-seq providing the expression of CXCR2 and CCR2 in shCtrl+vehicle, shNKX2-1+vehicle samples.

FIG. 42 shows the differential expression gene (DEG) analysis from scRNA-seq analysis providing the most upregulated and downregulated genes in macrophages.

FIGS. 43A-43B show unsupervised clustering of scRNA-seq data providing subdivision of infiltrated neutrophils into six distinct subpopulations (FIG. 43A) and in separate samples of shCtrl+vehicle, shNKX2-1+vehicle, and shNKX2-1+SB225002 cells (FIG. 43A).

FIG. 44 shows GO-BP enrichment analysis providing the biological processes in cluster 3 neutrophil population.

FIG. 45 shows the pseudo-time visualization providing the single-cell lineage order based on the gene expression profile from scRNA-seq analysis.

FIG. 46 shows the graphical representation of cell percentages according to neutrophil clusters with more than one read for the corresponding gene.

FIG. 47 shows the violin plot providing the gene expression score of upregulated cancer-promoting genes, expressed in shCtrl+vehicle, shNKX2-1+vehicle, and shNKX2-1+SB225002 infiltrated neutrophils.

FIG. 48 shows the ridgeline plot visualization of the expression distributions of the indicated differentially expressed cancer-promoting genes from cluster 3 across three samples (shCtrl+vehicle, shNKX2-1+vehicle, and shNKX2-1+SB225002 cells).

FIG. 49 shows the Venn diagram providing the numbers of NKX2-1-dependent genes involved in CXCR2-mediated signaling. The left circle denotes the upregulated genes in shNKX2-1+vehicle vs shCtrl+vehicle, and the right circle represents the downregulated genes in shNKX2-1+SB225002 vs shNKX2-1+vehicle samples. The overlapping genes are NKX2-1-regulated genes in the context of CXCR2-mediated signaling.

FIG. 50 shows the GO-BP enrichment analysis of the indicated groups of 101, 14, and 263 genes shown in FIG. 49.

FIG. 51 shows the pie chart representation providing the enriched GO-BP terms with the upregulated gene percentage obtained from scRNA-seq analysis of NKX2-1-low sample compared to the control group and RNA-seq from HL-60 cultured with conditioned medium from shNKX2-1/H1975 cells compared with shCtrl.

FIG. 52 shows the graphical representation of the enriched GO-BP analysis from scRNA-seq analysis of NKX2-1-low tumors compared to the control group and RNA-seq from HL-60 cultured with conditioned medium from shNKX2-1/H1975 cells compared with shCtrl.

FIGS. 53A-53B show that the networks engaged by NKX2-1-low tumor-activated neutrophils in in vitro and in vivo models. Tthe network analysis showing different genes involved in the enriched GO-BP analysis from scRNA-seq analysis of NKX2-1-low sample compared to the control group (FIG. 53A) and RNA-seq from HL-60 cultured with conditioned medium from shNKX2-1/H1975 cells compared with shCtrl (FIG. 53B).

### DETAILED DESCRIPTION OF THE INVENTION

Unless defined otherwise, all technical and scientific terms used herein have the same meaning as commonly understood by a person skilled in the art to which this invention belongs.

### Abbreviations

As used herein, the abbreviations have the definitions below:
NKX2-1: NK2 homeobox 1 or thyroid transcription factor-1
LUAD: Lung adenocarcinoma
TCGA: The Cancer Genome Atlas
TIMER: Tumor Immune Estimation Resource
EMT: Epithelial-mesenchymal transition
CXCR2: CXC motif chemokine receptor 2
SCLC: Small-cell lung carcinoma
NSCLC: Non-small cell lung carcinoma
EGFR: Epidermal growth factor receptor
EGFR-TKI: EGFR tyrosine kinase inhibitor
TAN: Tumor-associated neutrophils
TME: Tumor microenvironment
TF: Transcription factor
scRNA-seq: Single-cell RNA sequencing
IHC: Immunohistochemistry
qRT-PCR: Quantitative reverse transcription polymerase chain reaction
ChIP-qPCR: Chromatin immunoprecipitation quantitative polymerase chain reaction
GO: Gene Ontology
GO-BP: Gene Ontology-Biological Process
GO-MF: Gene Ontology-Molecular Function

As used herein, the term “patient” or “subject” refers to a human or an animal suffering from a cancer, including a human or an animal. In the present invention, the subject or patient is a human.

As used herein, the term “treat,” “treating,” or “treatment” refers to both therapeutic treatment and prophylactic or preventative measures, wherein the object is to prevent, slow down, and/or halt the development of wound or DFU. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function. Accordingly, the invention provides a pharmaceutical composition comprising a therapeutically effective amount of the preparation of inhibitor of CXCR2, and one or more pharmaceutically acceptable carriers.

The present invention provides the present invention provides a method for predicting clinical outcomes of a lung cancer patient, comprising providing a cancer cell sample of the patient, and detecting the expression level of NKX2-1 in the sample, wherein the clinical outcome is predicted as poor if the expression of NKX2-1 is low.

In one embodiment of the present invention, the lung cancer is lung adenocarcinoma (LUAD), particularly LUAD.

In another aspect, the present invention provides a method for treating lung adenocarcinoma (LUAD) in a patient, comprising administering to said patient an inhibitor of CXCR2 at a therapeutically effective amount.

In one example, the LUAD is a cancer with low expression of NKX2-1.

In one example, the inhibitor of CXCR2 is CXCL1, CXCL2, and CXCL5.

In the invention, the inhibitor of CXCR2 is a CXCR2 antagonist comprising SB225002, MK-7123, Cpd 19, and AZD5069.

SB225002 is a non-peptide CXCR2 antagonist, which inhibits IL-8 binding to CXCR2 with an IC50 of 22 nM. The SB225002 has the structure as follow:

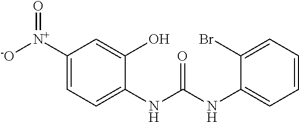

MK-7123, also known as Navarixin, which is an allosteric and orally active antagonist of both CXCR1 and CXCR2. It is used for reducing neutrophil chemotaxis and thus may alleviating airway inflammation in chronic obstructive pulmonary disease (COPD). The MK-7123 has the structure as follow:

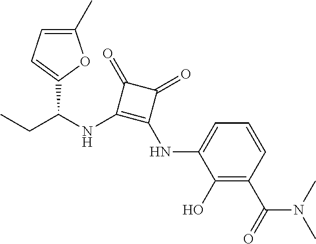 [SMILES: CC[C@@H](Nc1c(Nc2cccc(C(=O)N(C)C)c2O)c(=O)c1=O)c1ccc(C)o1]

AZD5069 is also a CXCR2 antagonist that inhibits ligand (IL-8 or GRO-α) induced cytosolic calcium increase, CD11b surface expression, adhesion and chemotaxis with an IC50 of 0.79 nM. The AZD5069 has the structure as follow:

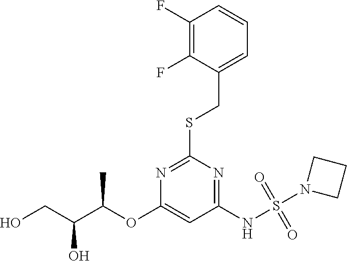 [SMILES: C[C@@H](Oc1cc(NS(=O)(=O)N2CCC2)nc(SCc2cccc(F)c2F)n1)[C@@H](O)CO]

NKX2-1 is a well-characterized pathological marker that delineates lung adenocarcinoma (LUAD) progression, where the progressiveness of LUAD relies on the immune tumor microenvironment through paracrine signaling. However, the involvement of NKX2-1 in modeling the tumor immune microenvironment is still unclear. In the present invention, the downregulation of NKX2-1 was observed in high-grade LUAD. Meanwhile, single-cell RNA sequencing and Visium in situ capturing profiling revealed the recruitment and infiltration of neutrophils in orthotopic syngeneic tumors exhibiting strong cell-cell communication through the activation of CXCLs/CXCR2 signaling. The depletion of NKX2-1 triggered the expression and secretion of CXCL1, CXCL2, CXCL3, and CXCL5 in LUAD cells. Chemokine secretion was analyzed by chemokine array and validated by qRT-PCR. ATAC-seq revealed the restrictive regulation of NKX2-1 on the promoters of CXCL1, CXCL2 and CXCL5. This phenomenon led to increased tumor growth, and conversely, tumor growth decreased when inhibited by the CXCR2 antagonist SB225002. The present invention unveils how NKX2-1 modulates the infiltration of tumor-promoting neutrophils by inhibiting CXCLs/CXCR2-dependent mechanisms. Hence, targeting CXCR2 in NKX2-1-low tumors is a potential antitumor therapy that may improve LUAD patient outcomes.

The present invention is further illustrated by the following examples, which are provided for the purpose of demonstration rather than limitation.

### EXAMPLES

### Materials and Methods

### Cell Culture

HCC827 (delE746_A750), H1975 (L858R/T790M), HCC827/GR, and H1975/AZDR lung cancer cells were obtained from Dr. Yu-Ting Chou's laboratory (National Tsing Hua University, Taiwan). HCC827/GR and H1975/AZDR cells were initially established by treating HCC827 and H1975 cell lines with the increased concentrations of gefitinib and osimertinib for 6 months, and the surviving cells were pooled together and cultured [60]. HL-60 (CCL-240) promyeoloblast cell line was obtained from the American Type Culture Collection (ATCC). These cells were cultured with RPMI-1640 medium (Thermo Fisher Scientific, Waltham, MA, USA) supplemented with 10% fetal bovine serum (FBS), 100 mg/mL streptomycin, and 100 U/mL penicillin. Lewis lung carcinoma (LL2) cell line was obtained from Dr. Nien-Jung Chen's laboratory (National Yang-Ming Chiao Tung University). These cells were maintained in DMEM medium supplemented with 10% FBS, 100 mg/mL streptomycin, and 100 U/mL penicillin. All cells were maintained at 37° C. with 5% CO₂ and were all tested negative for mycoplasma contamination. Gefitinib and osimertinib were purchased from Selleck Chemicals (Houston, TX, USA), and dissolved in dimethyl sulfoxide (DMSO; MP Biomedicals, Santa Ana, CA, USA) at a concentration of 10 mmol/L.

### Animal Experiments

All animals used in this invention were bred and maintained according to the Guidelines for Laboratory Animal Welfare in the Taipei Veterans General Hospital under the supervision of the Department of Medical Research of Taipei Veterans General Hospital (IACUC No. 2021-047 and 2022-097).

Female C57BL/6 mice were purchased from the Jackson Laboratory (Bar Harbor, ME, USA). Eight to ten-week-old mice were used in all the experiments. The mice were housed and maintained under specific-pathogen-free (SPF) conditions in an animal facility. In in vivo experiments, C57BL/6 mice and Lewis lung carcinoma (LL2) cell lines were used. CXCR2 inhibitor (SB225002) was dissolved in 1% DMSO, 20% polyethylene glycol 400, 5% Tween 80, and 74% ddH2O. SB225002 was administered at 10 mg/kg by intraperitoneal injection every other day. Control groups received solvent (1% DMSO, 20% PEG 400, and 5% Tween 80). On day 0, LL2 cells expressing luciferase reporter and eGFP (shCtrl and shNKX2-1) cells were collected and resuspended in PBS. For the subcutaneous tumor model, 100 μL cell suspension containing 5×10⁵ cells was injected subcutaneously into the flank region, while treatment started when the tumors were palpable. Mice were sacrificed on days 22-23. For the orthotopic lung cancer model, 20 μL cell suspension containing 5×10⁵ cells was injected through intrathoracic injection. The treatment started on day 1 and ended on day 5. Mice were sacrificed on day 7.

### Ethic Statements and Human Samples

The experimental procedures and protocols involving human samples were conducted according to the tenets of the Declaration of Helsinki and were approved by the Institutional Review Board of Taipei Veterans General Hospital (protocol no. 2020-04-009B and 2020-10-003B). Human tissue samples were obtained after taking informed consent from the patients.

Plasmids, shRNAs, and Cell Transfection

shCtrl (pLKO.1), human shNKX2-1 (TRCN0000020449_NM_003317 and TRCN0000020450_NM_003317), and mouse shNKX2-1 (TRCN0000862665 and TRCN0000086267) were purchased from Academia Sinica RNAi core (Taipei, Taiwan). NKX2-1 (pcDNA3.1 (+) wt TTF-1; 49989) overexpression plasmid, and pHAGE PGK-GFP-IRES-LUC-W (46793) plasmid were obtained from Addgene (Watertown, MA, USA). The lentivirus vector was co-transfected with packaging and envelope plasmids (psPAX2 and pMD2G) into HEK 293T cells to obtain lentivirus particles. Viral supernatants were collected 72 h after transfection, followed by ultracentrifugation at 82,700 g for 2 h. Cells were infected with lentivirus and 8 μg/ml polybrene (Sigma-Aldrich, St Louis, MO, USA) according to the instructions of Addgene (http://www.addgene.org/). Subsequently, the cells were selected with puromycin (2 μg/ml) to establish stable cell lines.

TransIT-LT1 Transfection Reagent (Mirus Bio, Madison, WI, USA) was used for transient transfection. All procedures were conducted according to the manufacturer's guidelines. Plasmids used in this invention are listed in Table 1. qRT-PCR and immunoblotting were used to validate the knockdown efficiency by shRNAs.

TABLE 1
List of plasmids

| Plasmids | Target sequence | Sequence ID No. | Catalog No. |
|---|---|---|---|
| pcDNA3.1(+) wt TTF-1 | CDS region: NM_003317 | | 49989 |
| p3xFLAG-Myc-CMV- | Vector backbone | | Addgene |
| 26 | | | |
| PHAGE PGK-GFP- | Empty backbone | | 46793 |
| IRES-LUC-W | | | |
| pLKO.1 empty | Empty backbone | | RNA icore |
| Human shRNA NKX2-1 | CGCTTGTAAATACCAGG | SEQ ID NO: 1 | TRCN0000020449 |
| pLKO.1 | ATTT | | |
| Human shRNA NKX2-1 | CGGCATGAACATGAGCG | SEQ ID NO: 2 | TRCN0000020450 |
| pLKO.1 | GCAT | | |
| Mouse shRNA NKX2-1 | CGGCATGAATATGAGTG | SEQ ID NO: 3 | TRCN000086265 |
| pLKO.1 | GCAT | | |
| Mouse shRNA NKX2-1 | GTTCTCAGTGTCTGACA | SEQ ID NO: 4 | TRCN0000086267 |
| pLKO.1 | TCTT | | |

RNA Extraction and qRT-PCR Analysis

The total RNA was exacted using RNeasy Mini Kit (QIAGEN, Hilden, Germany) and quantified by NanoDrop 2000 spectrophotometer (Thermo Fisher Scientific). According to the manufacturer's protocol, 1 μg of total RNA was subjected to first-strand complementary DNA synthesis using the SuperScript III Reverse Transcriptase Kit (Thermo Fisher Scientific). qRT-PCR reactions were performed using the SYBR Green kit in an ABI 7900 sequence detection system (Thermo Fisher Scientific) following the manufacturer's guidelines. The primers were designed using Primer Express Software v3.0.1 (Thermo Fisher Scientific) and are listed in Table 2. The specificity of all primers was computer-tested using BLAST (National Center for Biotechnology Information, Bethesda, MD, USA) by homology search with the human or mouse genome and further confirmed by dissociation curve analysis. The relative expression of mRNA was determined by the 2-AACT method and normalized to the endogenous expression of GAPDH mRNA.

TABLE 2
Primers for quantitative real-time PCR

| Species | Gene name | | Primers |
|---|---|---|---|
| Mouse | NKX2-1 | forward | 5′-CATGTCGATGAGTCCAAAGC-3′ (SEQ ID |
| | | | NO: 5) |
| | | reverse | 5′-CTCCATGCCCACTTTCTTGT-3′ (SEQ ID NO: |
| | | | 6) |
| | Cxcl1 | forward | 5′-TGCACCCAAACCGAAGTCAT-3′ (SEQ ID |
| | | | NO: 7) |
| | | reverse | 5′-ACTTGGGGACACCTTTTAGCA-3′ (SEQ ID |
| | | | NO: 8) |
| | Cxcl2 | forward | 5′-CTGCCAAGGGTTGACTTCAAGA-3′ (SEQ ID |
| | | | NO: 9) |
| | | reverse | 5′-GCTTCAGGGTCAAGGCAAACT-3′ (SEQ ID |
| | | | NO: 10) |
| | Cxcl3 | forward | 5′-CCAGACAGAAGTCATAGCCAC-3′ (SEQ ID |
| | | | NO: 11) |
| | | reverse | 5′-CTTCATCATGGTGAGGGGCTT-3′ (SEQ ID |
| | | | NO: 12) |
| | Cxcl5 | forward | 5′-TGCCCTACGGTGGAAGTCAT-3′ (SEQ ID |
| | | | NO: 13) |
| | | reverse | 5′-GCGAGTGCATTCCGCTTA-3′ (SEQ ID NO: |
| | | | 14) |
| | GAPDH | forward | 5′-GGAGGAACCTGCCAAGTATG-3′ (SEQ ID |
| | | | NO: 15) |
| | | reverse | 5′-TGGGAGTTGCTGTTGAAG-3′ (SEQ ID NO: |
| | | | 16) |
| | EpCAM | forward | 5′-AAGAACCGACAAGGACACGG-3′ (SEQ ID |
| | | | NO: 17) |
| | | reverse | 5′-TCTGATGGTCGTAGGGGCTT-3′ (SEQ ID |
| | | | NO: 18) |
| | E-cadherin | forward | 5′-ATGTCCTGGGCAGAGTGAGA-3′ (SEQ ID |
| | | | NO: 19) |
| | | reverse | 5′-TGGAGCTTTAGATGCCGCTT-3′ (SEQ ID |
| | | | NO: 20) |
| | Vimentin | forward | 5′-TTCTCTGGCACGTCTTGACC-3′ (SEQ ID NO: |
| | | | 21) |
| | | reverse | 5′-GCTTGGAAACGTCCACATCG-3′ (SEQ ID |
| | | | NO: 22) |
| | Fibronectin | forward | 5′-CAACCCTGGGTATGACACCG-3′ (SEQ ID |
| | | | NO: 23) |
| | | reverse | 5′-CCGCCTAAAGCCATGTTCCT-3′ (SEQ ID |
| | | | NO: 24) |
| Human | NKX2-1 | forward | 5′-AGCACACGACTCCGTTCTC-3′ (SEQ ID NO: |
| | | | 25) |
| | | reverse | 5′-GCCCACTTTCTTGTAGCTTTCC-3′ (SEQ ID |
| | | | NO: 26) |
| | CXCL1 | forward | 5′-CACCCCAAGAACATCCAAAG-3′ (SEQ ID |
| | | | NO: 27) |
| | | reverse | 5′-TAACTATGGGGGATGCAGGA-3′ (SEQ ID |
| | | | NO: 28) |
| | CXCL2 | forward | 5′-CACCTCAAGAACATCCAAAGTG-3′ (SEQ |
| | | | ID NO: 29) |
| | | reverse | 5′-GATTTTCTTAACCATGGGCG-3′ (SEQ ID |
| | | | NO: 30) |
| | CXCL3 | forward | 5′-GAGCGTCCGTGGTCACTGAA-3′ (SEQ ID |
| | | | NO: 31) |
| | | reverse | 5′-CCGGGGGACCTTACATTCAC-3′ (SEQ ID |
| | | | NO: 32) |
| | CXCL5 | forward | 5′-ACCACGCAAGGAGTTCATCC-3′ (SEQ ID |
| | | | NO: 33) |
| | | reverse | 5′-GGGGCTTCTGGATCAAGACA-3′ (SEQ ID |
| | | | NO: 34) |
| | EpCAM | forward | 5′-GCAGGGTCTAAAAGCTGGTGTT-3′ (SEQ ID |
| | | | NO: 35) |
| | | reverse | 5′-TCCCTATGCATCTCACCCATCT-3′ (SEQ ID |
| | | | NO: 36) |
| | E-cadherin | forward | 5′-ATTTTTCCCTCGACACCCGAT-3′ (SEQ ID |
| | | | NO: 37) |
| | | reverse | 5′-TCCCAGGCGTAGACCAAGA-3′ (SEQ ID |
| | | | NO: 38) |
| | Vimentin | forward | 5′-GGCGAGGAGAGCAGGATTTC-3′ (SEQ ID |
| | | | NO: 39) |
| | | reverse | 5′-AGTGGGTATCAACCAGAGGGA-3′ (SEQ ID |
| | | | NO: 40) |
| | Fibronectin | forward | 5′-ACCCCCACCAGCCTACTGAT-3′ (SEQ ID |
| | | | NO: 41) |
| | | reverse | 5′-ACTTGCTCCCAGGCACAGTG-3′ (SEQ ID |
| | | | NO: 42) |
| | ZEB1 | forward | 5′-AGCAGTGAAAGAGAAGGG-3′ |
| | | | (SEQ ID NO: 43) |
| | | reverse | 5′-GGTCCTCTTCAGGTGCCT-3′ |
| | | | (SEQ ID NO: 44) |
| | ZEB2 | forward | 5′-CCTCTGTAGATGGTCCAGAAGAA-3′ (SEQ |
| | | | ID NO: 45) |
| | | reverse | 5′-CTGTACCATTGTTAATTGCGGTC-3′ (SEQ |
| | | | ID NO: 46) |
| ChIP- | CXCL1 | forward | 5′-CCAGCCCCAACCATGCATAAAA-3′ (SEQ |
| qPCR | | | ID NO: 47) |
| | promoter | reverse | 5′-CTGTGAGAGGAGCGGAAGAGC-3′ (SEQ ID |
| | | | NO: 48) |
| | Control- | forward | 5′-ACTCTACCTGCACACTGTCCTA-3′ (SEQ ID |
| | CXCL1 | reverse | NO: 49) |
| | | | 5′-ATGACTGGAGAACATTTGAAAACA-3′ |
| | | | (SEQ ID NO: 50) |
| | CXCL2 | forward | 5′-ATTCCCGGAGCTCCAGATCG-3′ (SEQ ID |
| | promoter | reverse | NO: 51) |
| | | | 5′-AAGACAGTCAGACCCGGACG-3′ (SEQ ID |
| | | | NO: 52) |
| | Control- | forward | 5′-TGCTACAACAATGTTCAAAGTCA-3′ (SEQ |
| | CXCL2 | | ID NO: 53) |
| | | reverse | 5′-GCATTGGGATTTTATAGATTCTGGA-3′ |
| | | | (SEQ ID NO: 54) |
| | CXCL5 | forward | 5′-CATAGTGGTCAAGAGAGCG-3′ (SEQ ID NO: |
| | promoter | | 55) |
| | | reverse | 5′-GGGGAGAGATGAGTGTAGAT-3′ (SEQ ID |
| | | | NO: 56) |
| | Control- | forward | 5′-AAACATAACTTAGTGACAAGC-3′ (SEQ ID |
| | CXCL5 | | NO: 57) |
| | | reverse | 5′-CTCTGTACGATTTAAGTAACA-3′ (SEQ ID |
| | | | NO: 58) |

### Western Blotting

The cells were lysed in RIPA lysis buffer (Beyotime Institute of Biotechnology, Haimen, China) containing proteinase and phosphatase inhibitor cocktail (Sigma-Aldrich, St. Louis, MO, USA). The total protein concentration was determined by the Bradford assay (Bio-Rad Laboratories, Hercules, CA, USA). Equal protein concentrations were resolved by SDS-PAGE, transferred onto PVDF membranes (MilliporeSigma, Burlington, MA, USA), and blocked with 5% skimmed milk in Tris-buffered saline with Tween 20. The membranes were incubated overnight at 4° C. with primary antibodies listed in Table 3. On the next day, the membranes were incubated with HRP-conjugated secondary antibodies. Immunoblots were visualized using the Immobilon Western Chemiluminescent HRP Substrate (MilliporeSigma). The blots were tested with GAPDH or α-tubulin antibodies to confirm equal protein loading.

TABLE 3
Antibody for Western blot, ChIP-qPCR, and Flow cytometry

| Antibodies | Supplier | Species | Catalog No. |
|---|---|---|---|
| TTF-1 (NKX2-1) | Cell Signaling Technology (CST) | Rb-Mono | 12373 |
| E-cadherin (24E10) | Cell Signaling Technology (CST) | Rb-Mono | 3195 |
| EpCAM | Cell Signaling Technology (CST) | Rb-Mono | 2626 |
| Vimentin | Sigma | Mouse-Mono | V6630 |
| ZEB1 | Cell Signaling Technology (CST) | Rb-Mono | 3396 |
| Phosphor-EGFR | Cell Signaling Technology (CST) | Rb-Mono | 3777 |
| EGFR | Cell Signaling Technology (CST) | Rb-Mono | 4267 |
| Alpha-tubulin | Sigma | Mouse-Mono | T6074 |
| CXCR2 | GeneTex | Mouse-Mono | GTX631667 |
| CD66b | Abcam | Rb-Poly | ab197678 |
| Neutrophil Elastase | Abcam | Rb-Poly | ab68672 |
| CD11b | Invitrogen | Rb-Mono | 14-0112-82 |
| PerCP/Cyanine5.5 | Biolegend | Mouse-Mono | 155525 |
| CD170 (Siglec-F) | | | |
| Pacific Blue ™ CD11b | Biolegend | Mouse-Mono | 301316 |
| APC/Cyanine7 Ly-6G | Biolegend | Mouse-Mono | 127623 |
| PerCP Ly6-C | Biolegend | Mouse-Mono | 128027 |
| PE/Cyanine7, Ly-6G | Biolegend | Mouse-Mono | 127617 |
| BV421 Rat, Siglec-F | Biolegend | Mouse-Mono | 562681 |

### Human Chemokine Array

For secreted protein expression analysis, the Proteome Profiler Human Chemokine Array Kit (R&D Systems, Minneapolis, MN, USA) with catalog #: ARY017 was used according to the manufacturer's protocol.

### RNA-Seq Analysis

Following the manufacturer's instructions, the purified RNA was used to prepare the sequencing library by TruSeq Stranded mRNA Library Prep Kit (Illumina, San Diego, CA, USA). Briefly, mRNA was purified from 1 μg of total RNA by oligo (dT)-coupled magnetic beads and fragmented into small pieces under elevated temperature. The first-strand cDNA was synthesized using reverse transcriptase and random primers. The adaptors were ligated after the generation of double-stranded cDNA and adenylation of 3′ ends of DNA fragments. The products were enriched by PCR and purified with AMPure XP system (Beckman Coulter, Brea, CA, USA). The libraries were qualified by Qsep400 System (BiOptic Inc., New Taipei City, Taiwan) and quantified by Qubit 2.0 Fluorometer (Thermo Fisher Scientific). The qualified libraries were then sequenced on an Illumina NovaSeq 6000 platform with 150 bp paired-end reads generated by Genomics, BioSci & Tech Co., New Taipei City, Taiwan.

The low-quality reads from the raw data were removed using the fastp (version 0.20.0 software). The filtered reads were aligned to the reference genomes using HISAT2 (version 2.1.0). The software featureCounts (v2.0.1) in the Subread package was applied to quantify the gene abundance. Differentially expressed genes were identified by DESeq2 (version 1.28.0) [4] or EdgeR (version 3.36.0). The functional enrichment analysis of Gene Ontology (GO) terms was implemented in an R package clusterProfiler (version 4.0.0).

### Chromatin Immunoprecipitation Quantitative Real-Time PCR (ChIP-qPCR)

ChIP-qPCR was performed using a High Cell Number Chromatin Immunoprecipitation kit (Diagenode, Denville, NJ, USA). 1×10⁷ cells/ml resuspended in PBS were processed according to the manufacturer's instructions. Chromatin was sonicated using a Bioruptor sonicator (Diagenode) according to the manufacturer's protocol and examined with an electrophoresis assay for shearing optimization. Enriched DNA was quantified by performing real-time PCR using SYBR Green qPCR Master Mix (Thermo Fisher Scientific) in an ABI 7900 sequence detection system (Thermo Fisher Scientific) following the manufacturer's guidelines. The antibodies and primers used in ChIP-qPCR assays are listed in Tables 3 and 2. The enrichment signal was normalized to input DNA.

Assay for Transposase-Accessible Chromatin with Sequencing (ATAC-Seq)

ATAC-seq libraries were prepared by using an ATAC-seq kit (Diagenode; #C01080001) following the manufacturer's instructions. In brief, 5×10⁶ H1975 (shCtrl and shNKX2-1) cells were harvested, washed and the pellet was lysed. Nuclei were extracted, and tagmentation was performed according to the manufacturer's instructions. DNA was isolated by using a spin column and the transposase-processed DNA fragments were amplified by using the 2× High-Fidelity Master mix with 1 μl barcoded primers (#C01011035) for 13 cycles. AMPure XP beads (Beckman Coulter) were used to purify the DNA following the manufacturer's protocol. Qubit Flex Fluorometer was used to assess the DNA quality and integrity.

Sequencing was performed, and the fastq files were analyzed using the ENCODE ATAC-seq pipeline for single-end reads with default parameters. The genome fasta file was processed by the build_genome_data.sh script (supplied with the ATAC-seq pipeline). The output mapping and peak files of each sample were further analyzed. The resulting files were visualized using the IGV viewer [61]. MEME suites 5.5.4 was used to find the motif enrichment

### Tissue Microarrays and Immunofluorescence

LUAD tissue microarray slides (TMAs) were obtained from US Biomax (Rockville, MD, USA). LC641 microarray panel contained 64 cases of LUAD, while the LC10013c microarray panel contained 48 cases of LUAD with matched adjacent normal lung tissues. TMA immunostaining was performed according to the manufacturer's protocol. The antibodies used are listed in Table 3. The scores of immunoreactivity patterns of all tissues from TMA were examined at the Department of Pathology, Taipei Veterans General Hospital, under the supervision of the Department of Medical Research and Education of Taipei Veterans General Hospital.

For immunofluorescence, a total of 1×10⁵ cells were plated onto 35 mm dish. Following a 24-hour incubation period, the cells were fixed using 4% paraformaldehyde in PBS for 15 minutes and permeabilized with 0.5% Triton™ MX-100 in PBS for 15 minutes at room temperature. Subsequently, the cells were blocked with 10% BSA for 1 hour at room temperature. After each step, the cells were washed thrice with PBS. The cells were then subjected to overnight incubation with NKX2-1 antibody at 4° C. Following this, the cells were exposed to a fluorescent-labeled secondary antibody for 1 hour along with DAPI (Sigma-Aldrich) at room temperature. Finally, the cells were mounted using a mounting solution and examined under a microscope.

### Chemotaxis Assay and Co-Culture Experiment

8 μm pore size FluoroBlok cell culture inserts (Corning Inc., Corning, NY, USA) were used to perform a chemotaxis transwell migration assay. HL-60 cells were seeded in the transwell's upper chamber, and the medium from LUAD cells (HCC827 and H1975) was added to the lower chamber. After 16 h incubation, the migrated cells were fixed with methanol and stained with propidium iodide. The stained cells were viewed under a microscope and further quantified using ImageJ.

For the co-culture experiment, the medium from shCtrl/H1975 or shNKX2-1/H1975 cell culture was used to culture HL-60 cells for 5 days. The medium was changed every other day.

### Flow Cytometry Analysis

Lungs containing tumor nodules were collected after mice were sacrificed. The mouse lung tissues were dissected and cut into small pieces. Dissected tissues were incubated with 1 mg/mL collagenase type I and DNase 1 in RPMI 1640 basic medium for 1 h at 37° C. After incubation, the digested tissue was passed through a 70 μm cell strainer to improve cell dissociation.

Red Blood Cell Lysis Buffer (154 mM NH₄Cl, 10 mM KHCO₃, 0.1 mM EDTA, pH 7.4) was added to the single-cell suspension to lyse red blood cells. The digested cells were washed three times and resuspended in PBS. 1× 10⁶ cells/mL were stained with 1 μl fluorescence-conjugated antibodies (BD Biosciences, Franklin Lakes, NJ, USA; dilution 1:100) for 30 min in 100 μl PBS at 4° C. The cells were washed twice before flow cytometry analysis. Antibodies used are Pacific Blue- labeled mouse anti-human CD11b Antibody, PerCP/Cyanine5.5-labeled rat anti-mouse CD170 (Siglec-F), APC/Cyanine7-labeled rat anti-mouse Ly-6G, PerCP-labeled rat anti-mouse Ly-6C, APC/Cyanine7-labeled mouse anti-human CD11b, Human Integrin alpha M/CD11b PE-conjugated mouse anti-mouse (R&D; FAB16991P), PE/Cyanine7-labeled mouse anti-mouse Ly-6G, BV421-labeled rat anti-Mouse Siglec-F. Data acquisition was performed on BD FACSCanto II System Flow Cytometer (BD Biosciences) and was analyzed by FlowJo Software.

Single-Cell RNA Sequencing (scRNA-Seq)

scRNA-seq libraries of LL2 expressing luciferase reporter- and eGFP cells (shCtrl, shNKX2-1, and shNKX2-1 treated with CXCR2 antagonist) were generated by using a Chromium Controller instrument (10× Genomics, Pleasanton, CA, USA) and Chromium Single Cell 3′ Reagent Kits v2 according to the manufacturer's instructions. scRNA-Seq reads were aligned to the mouse reference genome dataset and quantified using Cell Ranger version 6.1.0 (10× Genomics default pipeline. Raw count matrix files were imported into the R package Seurat version 4.1.1 for downstream processing. First, we used the package SoupX to remove ambient RNA contamination from raw scRNA-seq data. Cells with a gene number more than 500 and a mitochondrial gene proportion <0.2 were selected for downstream analysis. The matrices used regularized negative binomial regression to normalize UMI count data by “SCTransform” (v2) and regress out the percentage of mitochondrial genes in each cell. We removed the doublet by “Doubletfinder” (estimate doublet ratio by 8% % for every additional 1000 cells). After quality control and “SCTransform” (v2) normalization, the subsequent steps are based on the standard process of the “Seurat” package. 3000 feature genes are selected to integrate data. For the integrated data, “RunPCA” is used for data dimension reduction. The uniform manifold approximation and projection algorithm is used for visualizing the dimension reduction data by the “RunUMAP” function. Then we clustered cells by “FindNeighbors” and “FindClusters” functions based on the Leiden algorithm. FindConservedMarkers was used for cell type annotation based on gene markers. The findmakers function was used to find the differential gene analysis between each group. We used R package iTALK (0.1.0) to perform the crosstalk between tumor and immune cells.

### Spatial Transcriptomics

Formalin-fixed paraffin-embedded (FFPE) samples passed the RNA quality control (DV200>50%) on shCtrl/LL2 tumors (shCtrl), and shNKX2-1/LL2 tumors. They were further processed for Visium in situ capturing construction and sequencing. The tissue was prepared according to the Visium CytAssist Spatial Gene Expression for FFPE-Tissue Preparation Guide (#CG000618; 10× Genomics, Pleasanton, CA, USA). The sequencing was performed by Genomics, BioSci & Tech Co. (New Taipei City, Taiwan). The Space Ranger pipeline v2.0.1 (10× Genomics) and the mm10-2020-A reference were used to process FASTQ files. The sequencing results were guaranteed to be accurate as follows: For shCtrl sample: Number of spots under tissue: 2642; mean reads per spot: 80303; median genes per spot 8365; number of reads: 212,160,122; valid barcodes: 99.2%; valid UMIs: 100%; sequencing saturation: 26.7%. For shNKX2-1 sample: Number of spots under tissue: 1973; mean reads per spot: 89390; median genes per spot 8719; number of reads: 176,365,674; valid barcodes: 99.2%; valid UMIs: 100%; sequencing saturation: 26.7%. UMAP and violin plots were run and plotted using Loupe Browser (10× genomics, Pleasanton, CA, USA). Trajectory analysis and pathway enrichment analysis were performed and plotted using Partek flow software (Partek Incorporated, Chesterfield, MO, USA).

### Statistical Analysis

Statistical analyses of data were presented as mean±standard deviation by using Microsoft Excel and GraphPad Prism. Two-tailed unpaired Student's t-test was used for the two-group comparison. One-way ANOVA was used for multiple-group comparisons. Survival curves were plotted using the Kaplan-Meier method and assessed using a log-rank test. The criterion for significance was considered p<0.05.

### Results

Low Expression of NKX2-1 is Associated with Aggressive LUAD

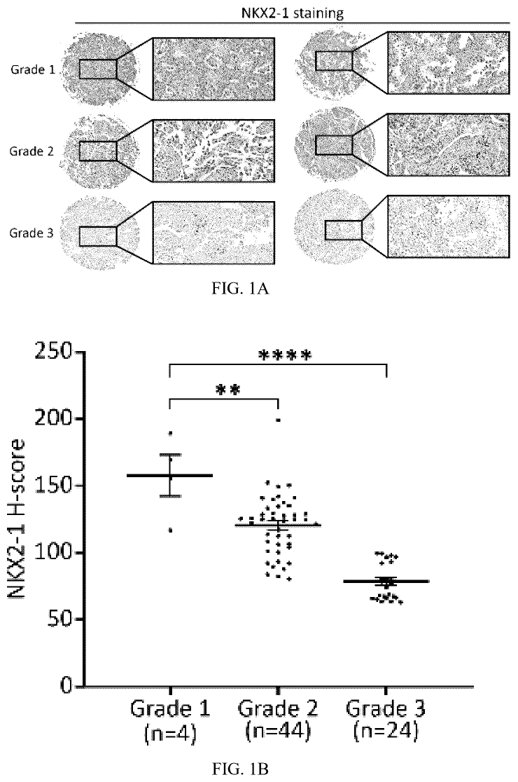

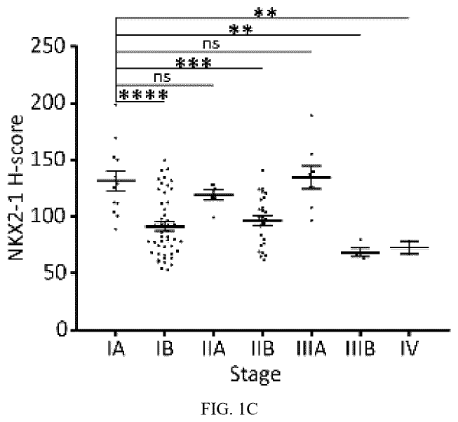

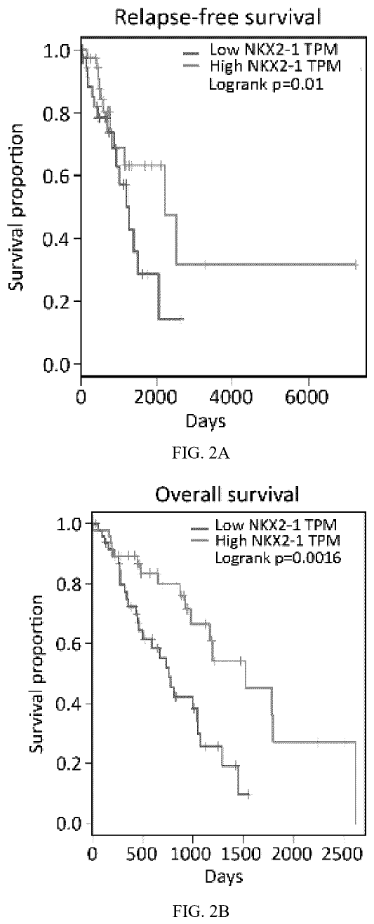

Downregulation of NKX2-1 is known to be associated with poorly differentiated lung adenocarcinoma (LUAD) tumors exhibiting high metastatic potential, and conversely, NKX2-1 is upregulated in less malignant well-differentiated LUAD tumors [22]. To validate the effects of NKX2-1, we investigated the protein expression levels of NKX2-1 across different grades of LUAD by conducting immunohistochemistry (IHC) staining on LUAD tissue microarray. As can be seen on the representative IHC images (FIG. 1A) or from quantification of the IHC staining (FIG. 1B), the protein expression of NKX2-1 in grade 3 LUAD tissues is markedly lower than in grade 1 and 2 tissues. Moreover, quantification of IHC staining based on LUAD staging showed that stage IIIB and stage IV LUAD tissues were characterized by low expression of NKX2-1 compared to stage IA, IB, IIA, IIB, and IIIA LUAD tissues (FIG. 1C). Furthermore, the survival analysis using The Cancer Genome Atlas (TCGA) dataset showed that high NKX2-1 expression levels correlated with better relapse-free survival and overall survival outcome in LUAD (FIGS. 2A and 2B).

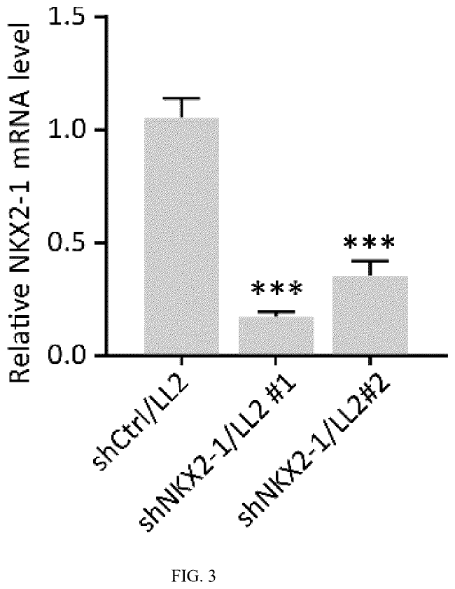

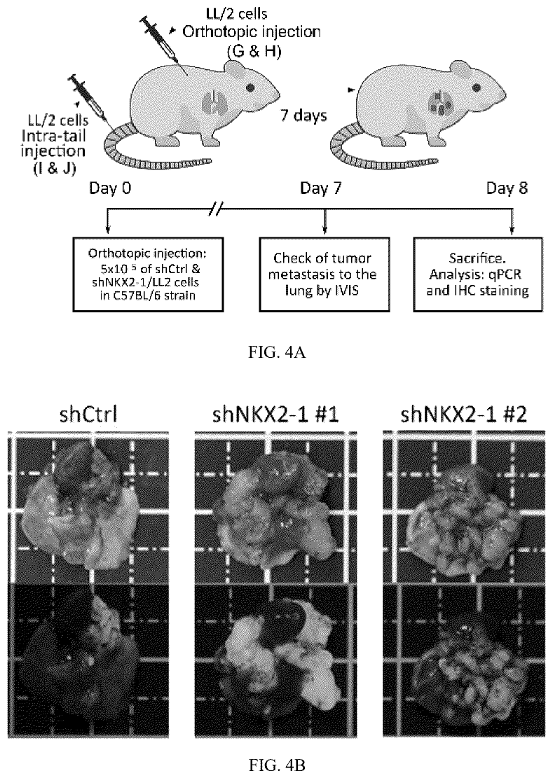

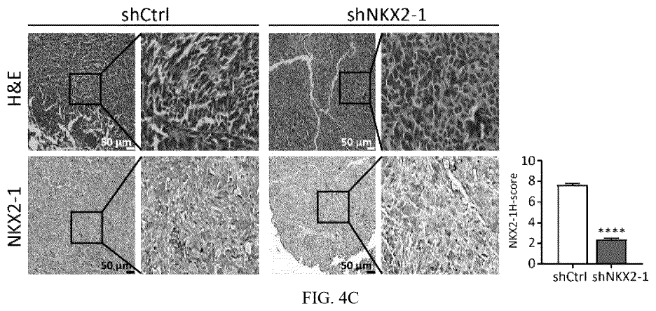

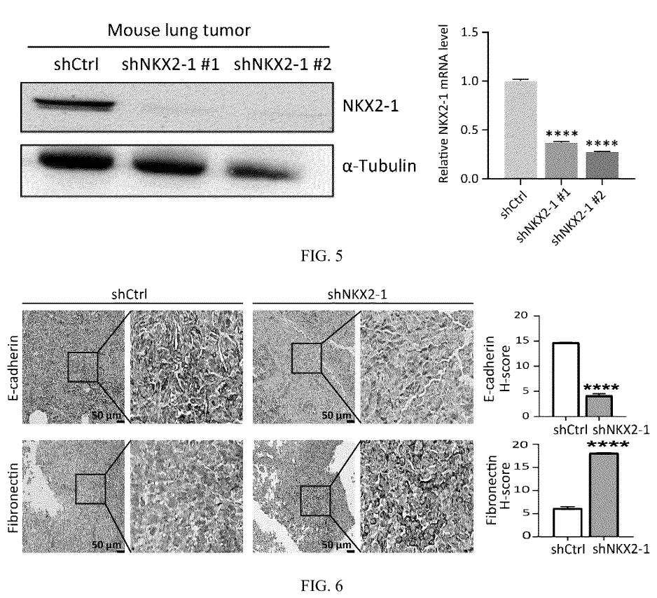

In this invention, we sought to explore the regulatory role of NKX2-1 in in vivo tumorigenicity by using both the orthotopic LUAD mouse model and the experimental model of LUAD distal metastasis. NKX2-1 was knocked down in murine Lewis lung carcinoma cells (LL2) expressing luciferase reporter and eGFP. qRT-PCR showed the knockdown of NKX2-1 in LL2 (shNKX2-1/LL2) cells with 60-70% efficiency compared to scrambled control shRNA (shCtrl/LL2) cells (FIG. 3). These cells were subjected either to orthotopic implantation to the lung or intra-tail injection (FIG. 4A). The orthotopically implanted shNKX2-1/LL2 cells generated tumors characterized by increased growth in the recipient mice compared to the shCtrl/LL2 implanted cells (FIG. 4B). IHC staining of NKX2-1 was carried out in the excised tumor tissues to validate the expression level of NKX2-1, and it showed low levels of NKX2-1 in shNKX2-1/LL2-derived tumor cells when compared with the shCtrl/LL2-derived tumor cells (FIG. 4C). Additionally, immunoblotting and qRT-PCR were also performed to validate the expression of NKX2-1 in the excised tumor tissues (FIG. 5). To validate prior studies that showed the regulatory role of NKX2-1 in epithelial-mesenchymal transition (EMT) [4], IHC staining of some EMT markers were performed on the excised tumor tissues. The results showed the upregulation of fibronectin, and the downregulation of E-cadherin in the tumors derived from shNKX2-1/LL2 cells when compared to shCtrl/LL2 cells. This indicated the induction of EMT after NKX2-1 knockdown (FIG. 6).

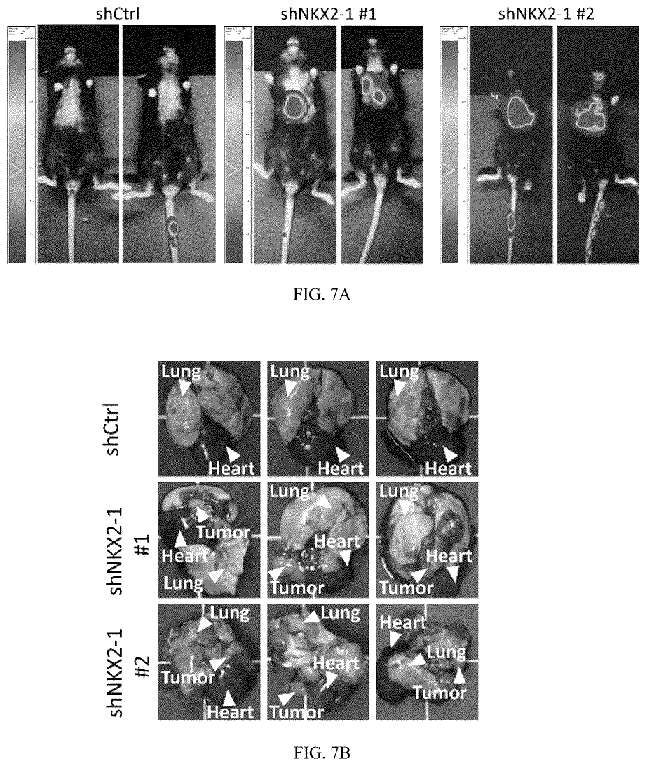

To demonstrate the metastatic potential in the orthotopic model, we intravenously injected shCtrl/LL2 or shNKX2-1/LL2 cells into the mice via the tail vein. The mice injected with shNKX2-1/LL2 cells exhibited a significant increase of luciferase signal at the upper torso compared to the shCtrl/LL2-injected mice (FIG. 7A). This result was in line with the considerable tumor nodules observed in the lungs of shNKX2-1/LL2 but not shCtrl/LL2-injected mice (FIG. 7B). These results indicated that low expression of NKX2-1 in LUAD contributed to increased tumor growth (FIG. 4B) and higher metastasis potential (FIG. 7A). In summary, the clinical data, cellular models, and animal experiments demonstrated that low expression of NKX2-1 was associated with features related to advanced cancer status and worse clinical outcomes.

NKX2-1 Expression Negatively Correlates with Neutrophil Infiltration

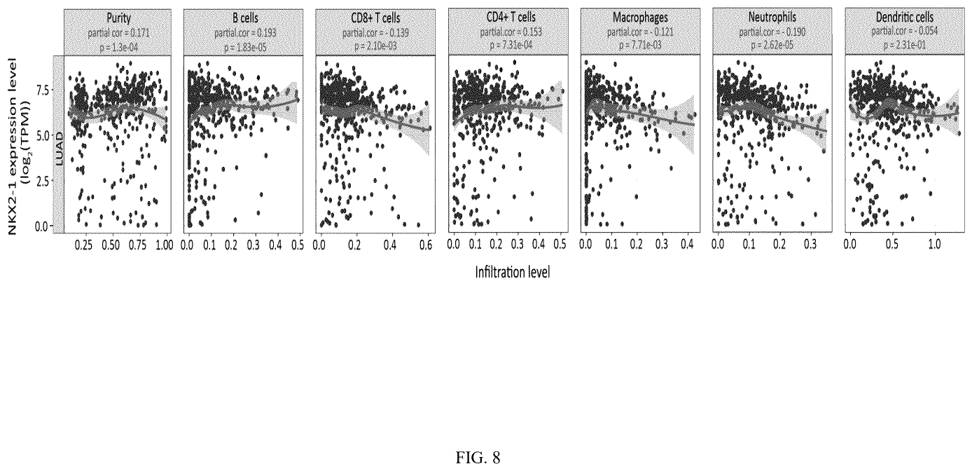

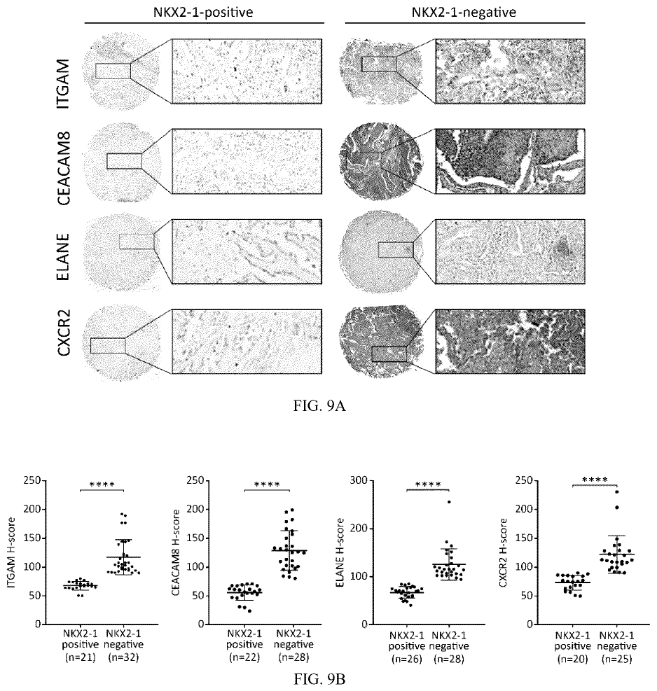

The tumor microenvironment (TME) can affect clinical outcomes due to the ability of the innate or adaptive immune cells to exert either tumor-promoting or tumor-suppressing effects [23]. The most abundant immune cells that infiltrate tumors are neutrophils and macrophages, which may exert pro-tumorigenic effects by driving angiogenesis, extracellular matrix remodeling, metastasis, and immunosuppression [24]. Therefore, we aimed to elucidate the impact of NKX2-1 on the complex interactions between malignant and immune cells within the TME. Tumor Immune Estimation Resource (TIMER) dataset was used to systematically analyze the correlation between NKX2-1 differential expression and immune infiltrations. The analysis showed that the infiltration of CD8+ T cells, macrophages, neutrophils, and dendritic cells negatively correlated with NKX2-1 expression levels in LUAD (FIG. 8). Given that the correlation with neutrophil infiltration was the most statistically significant and given the growing evidence of its oncogenic role in LUAD [8, 18, 23], we analyzed the expression of a set of neutrophil markers such as ITGAM, CEACAM8, ELANE, and CXCR2 by IHC staining of NKX2-1-negative and positive LUAD tissues. The representative images (FIG. 9A) and the quantification of the IHC staining (FIG. 9B) showed an upregulation of these markers in NKX2-1-negative LUAD tissues compared to NKX2-1-positive tissues.

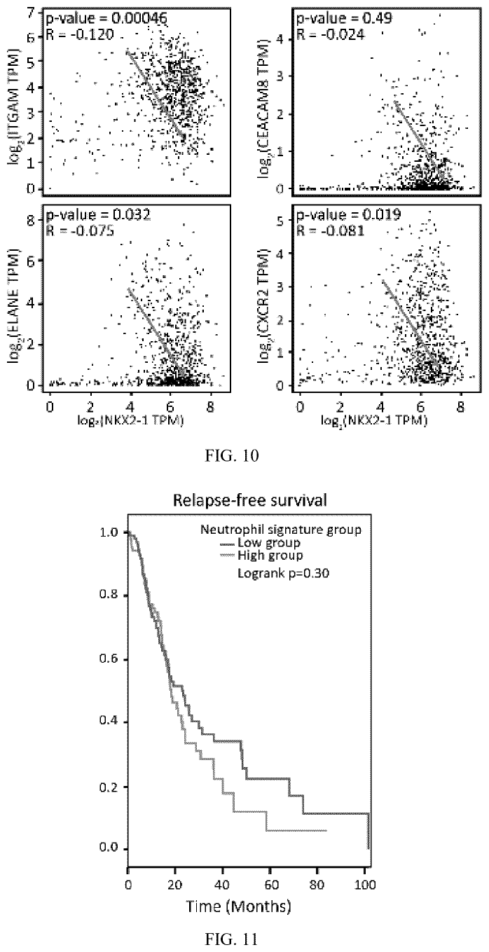

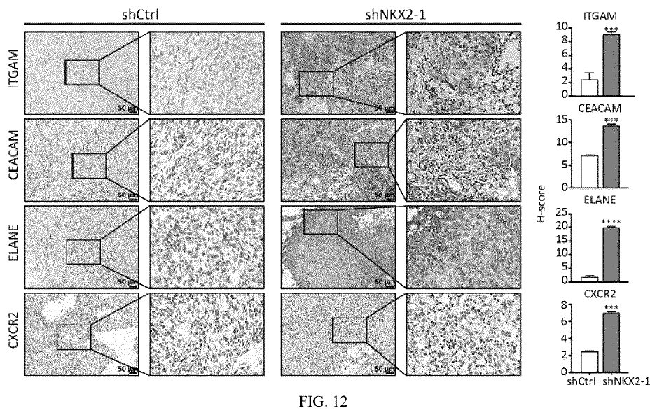

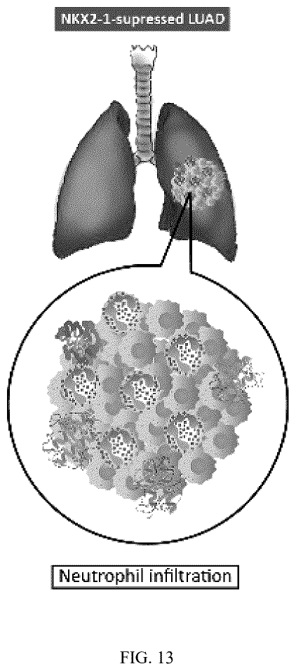

Furthermore, TCGA dataset analysis confirmed the negative correlation of NKX2-1 expression with the selected neutrophil markers (ITGAM, CEACAM8, ELANE, and CXCR2) in LUAD patients (FIG. 10). In addition, the survival analysis showed that low infiltration of neutrophils in relapse-free LUAD patient tumors was associated with better survival outcomes (FIG. 11). Moreover, IHC staining of the tumor tissues derived from shNKX2-1/LL2 cells orthotopically implanted into mouse lungs also showed elevated expression of ITGAM, CEACAM8, ELANE, and CXCR2 as compared to the control (FIG. 12). In conclusion, our data suggest that low expression of NKX2-1 correlates with high neutrophil infiltration and the abundance of neutrophil infiltration predicts poor clinical outcomes for LUAD patients (FIG. 13).

### NKX2-1-Low Tumors Promote Neutrophil Infiltration Via CXC Chemokines

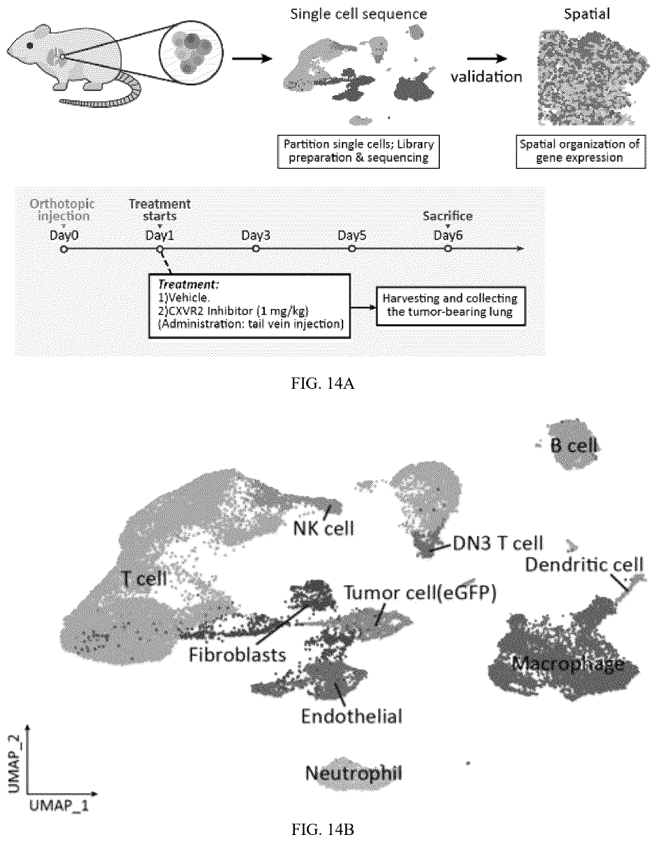

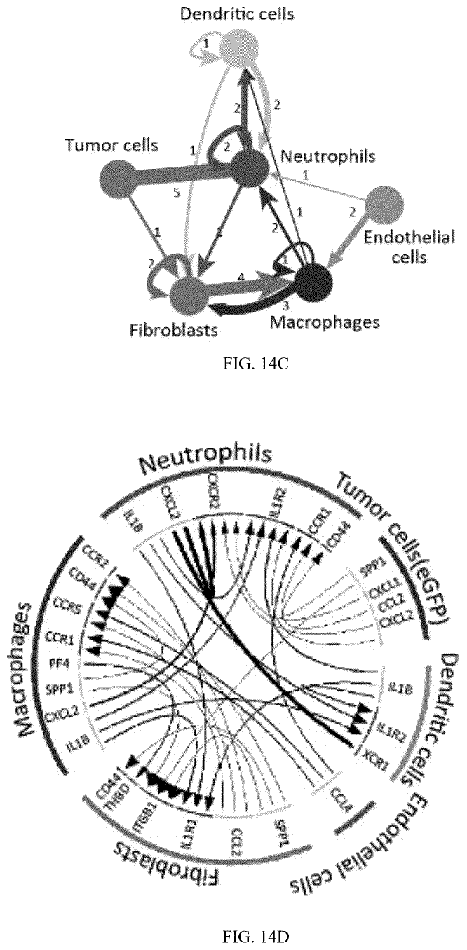

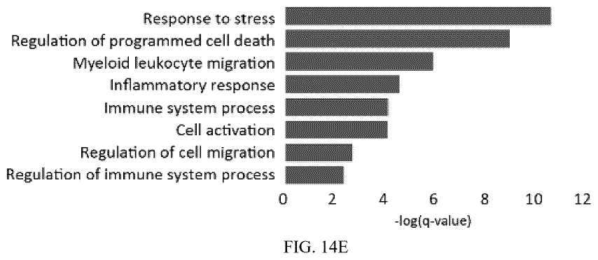

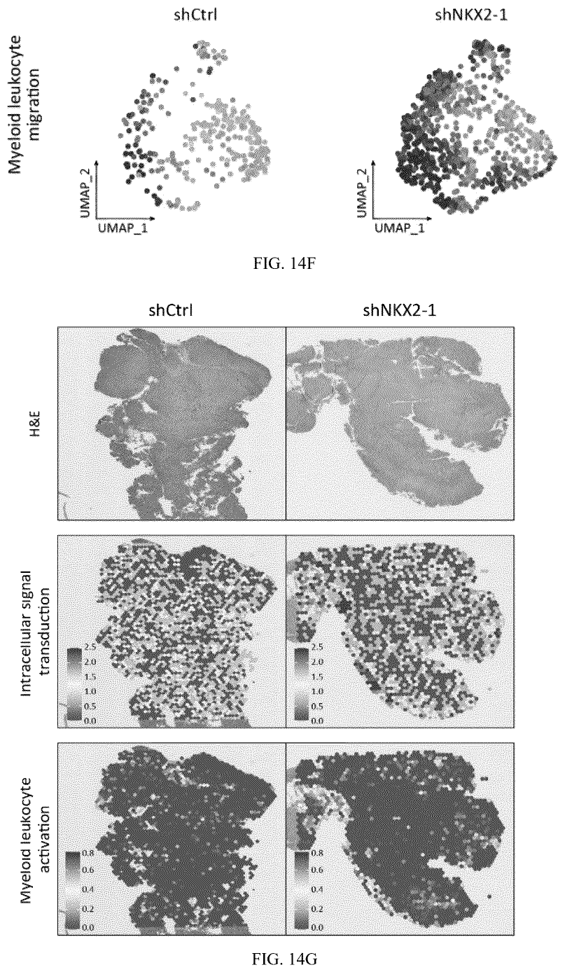

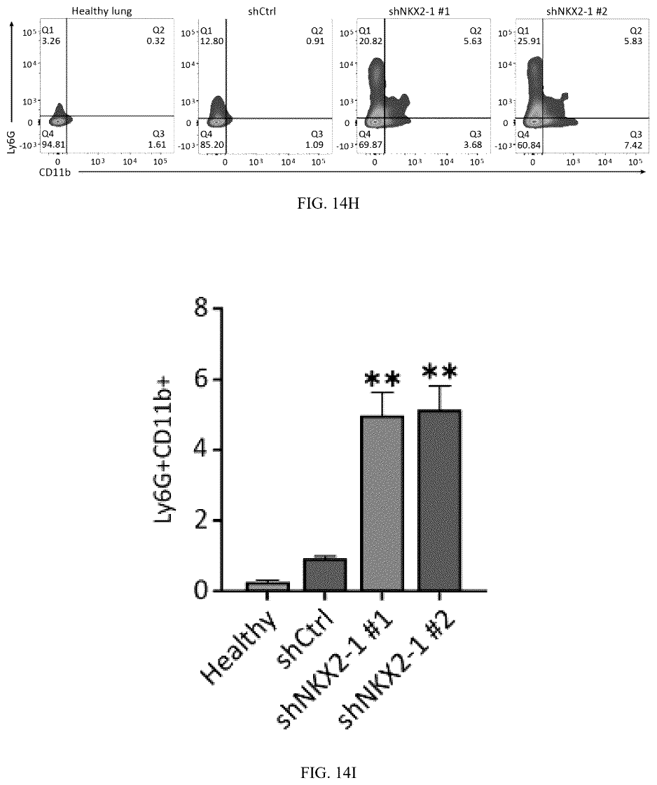

Chemokines secreted by both tumor cells and TME play a central role in regulating the recruitment of immune cells such as neutrophils [26]. Notably, the infiltration of tumor-promoting neutrophils leads to increased tumor progression and is generally associated with high-grade aggressive tumors [27]. Given that this observation is not always the same in all tumors due to their striking heterogeneity, we performed single-cell RNA sequencing (scRNA-seq) and Visium in situ capturing profiling on shCtrl/LL2 and shNKX2-1/LL2-derived tumor tissues to comprehensively characterize the infiltrated immune cells upon NKX2-1 knockdown at high resolution. For scRNA-seq, shCtrl/LL2 and shNKX2-1/LL2 cells were orthotopically implanted into mouse lungs, and the harvested tumors were dissociated into viable single cells (FIG. 14A).

To further characterize the distinct cell types from shCtrl/LL2 and shNKX2-1/LL2-derived tumors, preliminary clustering was applied to all the cells by using uniform manifold approximation and projection (UMAP) analysis, and FindAllMarkers package was used to find the different markers in each cluster (FIG. 14B). Meanwhile, the crosstalk between tumor cells and various cell types of the TME was further analyzed by using iTALK R package. The results indicated a significantly strong interaction of tumor cells with neutrophils (FIG. 14C). By further analysis of the cell-cell interactions, we found that the communication between tumor cells and neutrophils could occur through CXCLs/CXCR2 signaling activation (FIG. 14D). The enrichment analysis of gene ontology biological processes (GO-BP) showed enrichment in response to stress, regulation of programmed cells death, myeloid leukocyte migration, inflammatory response terms (FIG. 14E). To further visualize the enriched GO term related to myeloid cells, UMAP analysis revealed increase of myeloid leukocyte migration in NKX2-1 knocked down cells from our scRNA-seq (FIG. 14F), suggesting increased myeloid cells migration to shNKX2-1/LL2 tumors when compared to shCtrl tumors. To further validate the infiltration of immune cells in NKX2-1-low tumors, we performed a spatially resolved transcriptomics analysis with Visium in situ capturing profiling of shCtrl/LL2 tumors (shCtrl) and shNKX2-1/LL2 tumors was performed. Tissues were collected with a capture area of 8×8 mm, and ~5,000 gene expression spots were chosen (FIG. 14G). The results also showed an increase in myeloid leukocyte activation and intracellular signal transduction upon NKX2-1 downregulation (FIG. 14G). To validate the infiltration of neutrophils, we applied flow cytometry analysis to identify CD11b+Ly-6G+ cells, commonly recognized as tumor-associated neutrophils (TANs) [33, 34]. We observed a significant increase in CD11b+Ly-6G+ neutrophils in tumor-bearing lungs from the recipient mice intravenously injected with either clone of shNKX2-1/LL2 cells, compared to control mice transplanted with shCtrl/LL2 cells or the healthy lungs (FIGS. 14H and 14I). Altogether, our findings demonstrated an increase in neutrophil infiltration within NKX2-1-low tumors, potentially mediated by the activation of cell surface receptor signaling pathways.

### NKX2-1 Downregulation Affects the Expression of CXC Chemokines

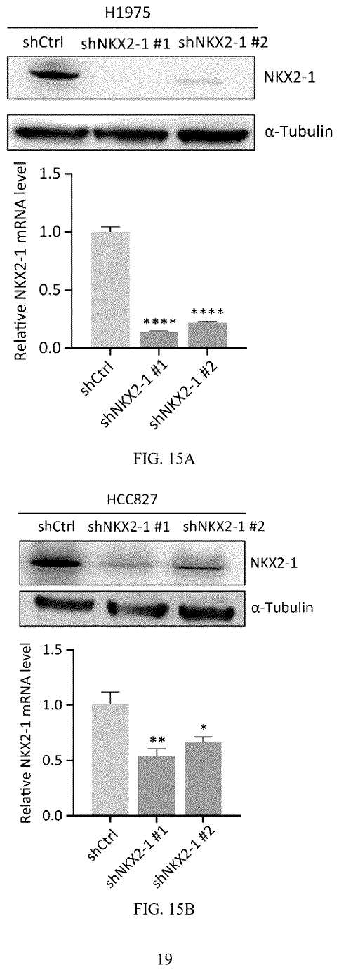

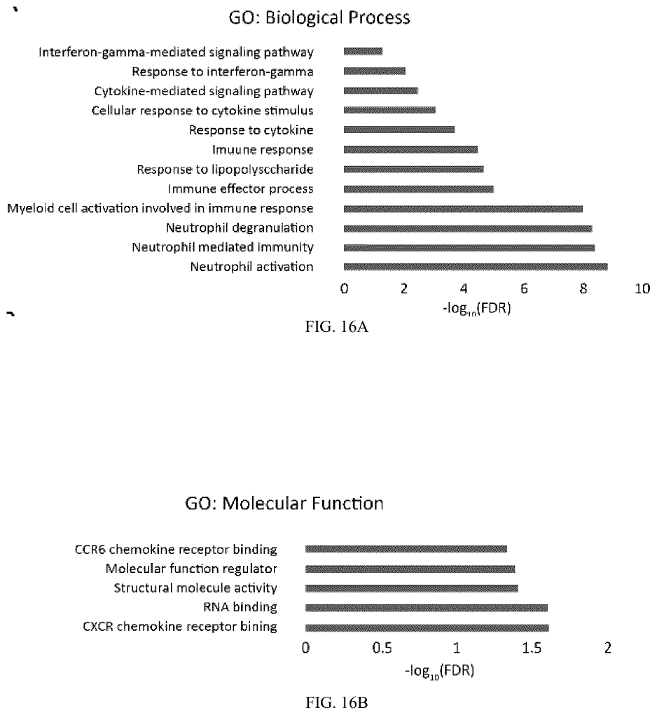

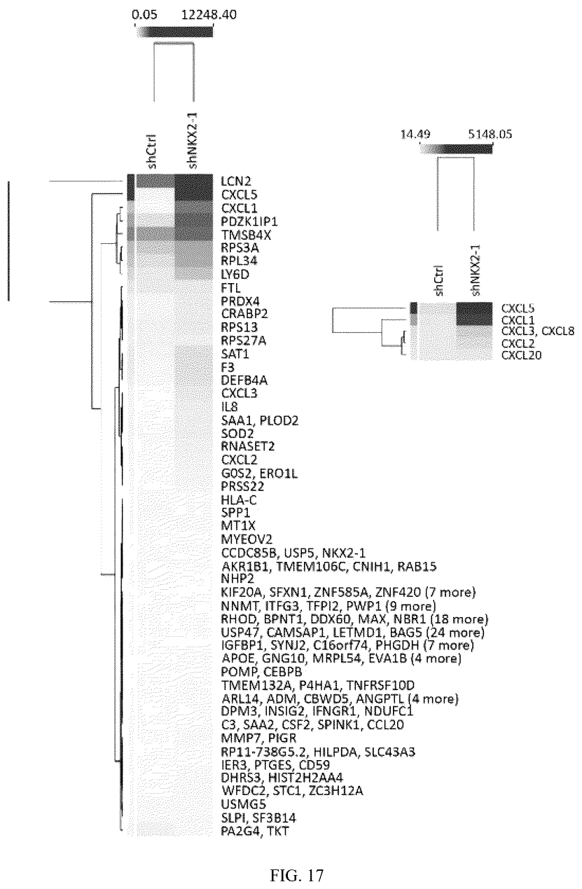

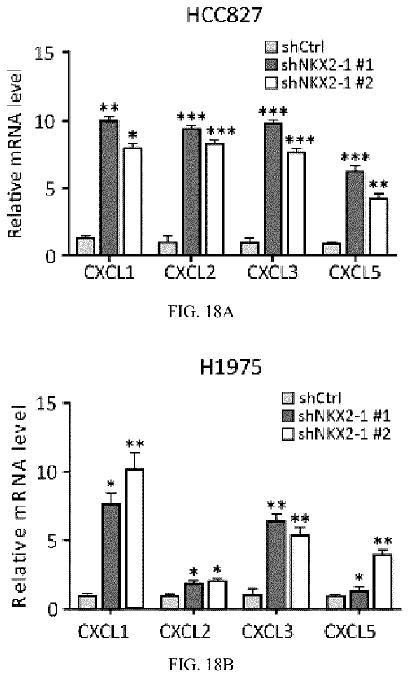

Having illustrated the role of CXC chemokines in mediating cell-cell communication between tumor cells and neutrophils by scRNA-seq analysis, we further proceeded to assess the regulatory role of NKX2-1 on CXC chemokine expression and secretion in human LUAD cell culture model. We efficiently knocked down NKX2-1 expression in NKX2-1-high LUAD cell lines (HCC827 and H1975) using shRNA constructs (FIGS. 15A and 15B). RNA-seq analysis was performed on HCC827 cells subjected to shRNA-mediated NKX2-1 knockdown. Functional enrichment analysis of the genes positively regulated by NKX2-1 knockdown revealed that the most enriched GO-BP terms were related to neutrophil activation, neutrophil-mediated immunity, and myeloid cell activation involved in immune response (FIG. 16A). Meanwhile, the most enriched GO molecular function (GO-MF) terms were related to CXCR chemokine receptor binding, and CCR6 chemokine receptor binding (FIG. 16B). These cell culture model observations on the role of CXC chemokines in neutrophil recruitment were consistent with our in vivo mouse model upon the functional elimination of NKX2-1. More specifically, our RNA-seq analysis identified the upregulation of several neutrophil chemotactic genes such as CXCL1, CXCL2, CXCL3, CXCL5, and CXCL8 upon NKX2-1 knockdown (FIG. 17). Among them, CXCL8 has been extensively characterized as a mediator of neutrophil mobilization and attraction [29]; therefore, we decided to explore the role of other CXC chemokines in NKX2-1-low tumors. qRT-PCR analysis was carried out to validate the RNA-seq data, and it confirmed that the knockdown of NKX2-1 by two shRNAs contributed to the upregulation of CXCL1, CXCL2, CXCL3, and CXCL5 mRNA levels in HCC827 and H1975 LUAD cells (FIGS. 18A and 18B).

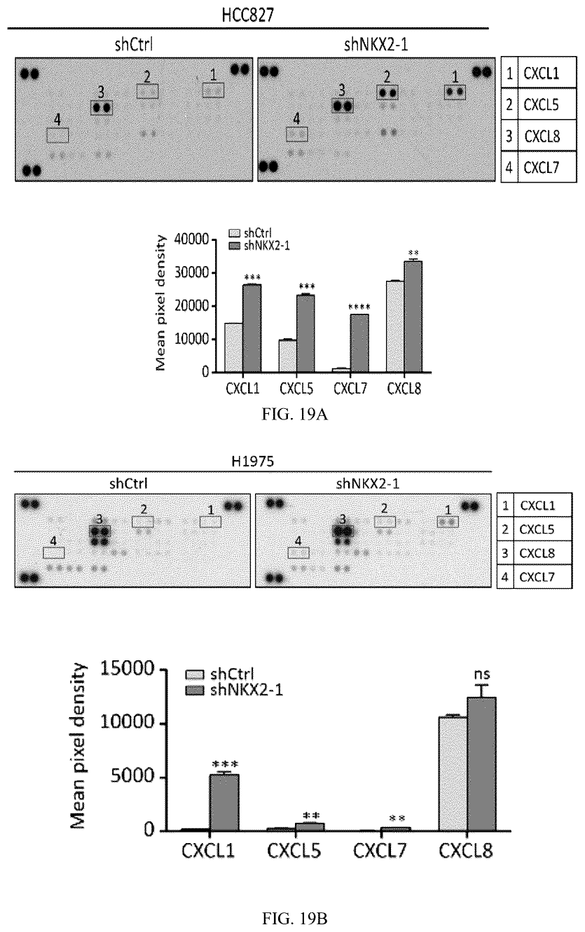

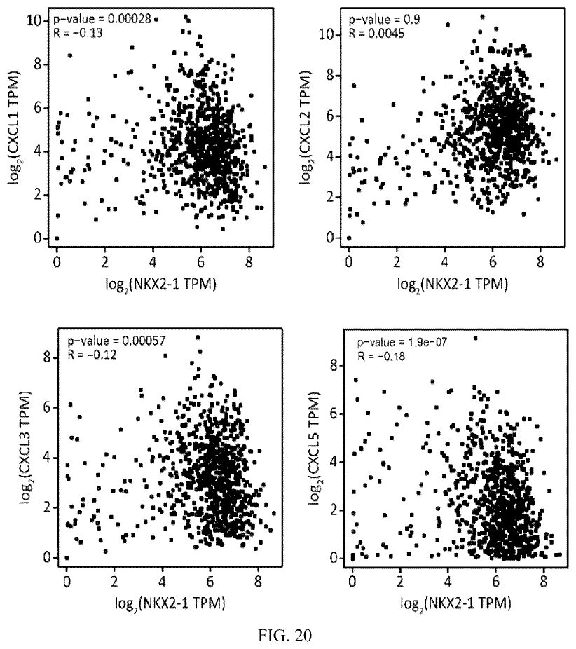

Furthermore, as was shown by the chemokine array, the knockdown of NKX2-1 in HCC827 cells led to increased secretion of CXCL1, CXCL5, CXCL7, and CXCL8 (FIG. 19A). Meanwhile, the knockdown of NKX2-1 in H1975 cells led to increased secretion of CXCL1, CXCL5, and CXCL7 with no significant secretion of CXCL8 compared to shCtrl (FIG. 19B). TCGA dataset analysis also indicated a negative correlation between the expression of NKX2-1 and CXCL1, CXCL3, and CXCL5. However, no correlation was observed between the expression of CXCL2 and NKX2-1 (FIG. 20). To summarize, our data demonstrated the role of NKX2-1 as a suppressor of CXC chemokine expressions and secretions in LUAD cells.

### NKX2-1 Suppresses the Chromatin Accessibility of CXC Chemokine Genes at the Promoter Regions

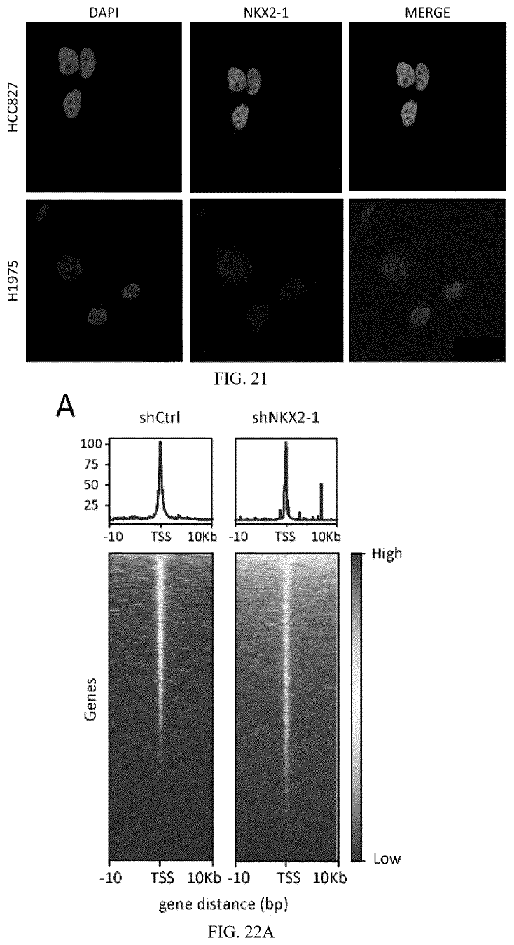

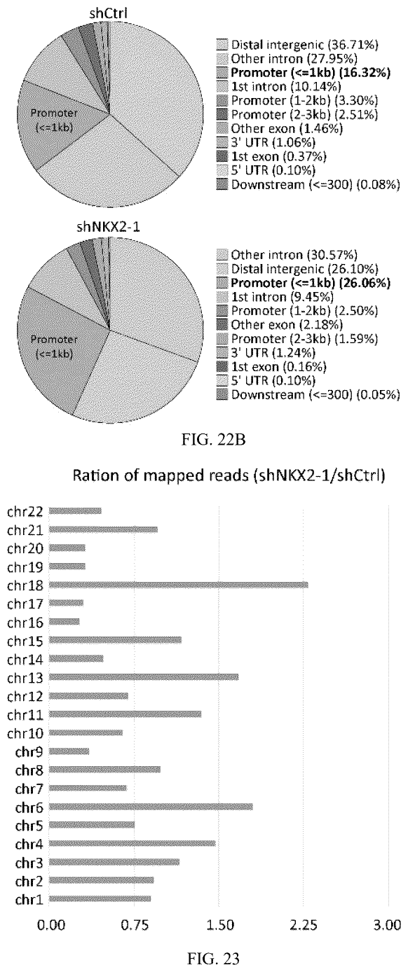

Following the observations that NKX2-1 suppresses the expression of various CXC chemokines in LUAD cells, we sought to elucidate the mechanism involved in NKX2-1-mediated suppression of CXC chemokine expression. NKX2-1 is a homeobox transcription factor that activates the transcription of thyroid and lung-specific genes [30], we confirmed the predominant localization of NKX2-1 in HCC827 and H1975 cells in the nucleus by immunofluorescence staining (FIG. 21). Therefore, we hypothesized that NKX2-1 could exert its action by affecting the chromatin structure. The open chromatin regions in shCtrl/H1975 and shNKX2-1/H1975 cells were sequenced by an assay for transposase-accessible chromatin using sequencing (ATAC-seq), a sequencing method based on the insertion of sequencing adapters by hyperactive Tn5 transposase [31]. To evaluate the effect of NKX2-1 on the overall pattern of chromatin accessibility, we estimated the abundance of reads with increased and decreased accessibility in shCtrl/H1975 and shNKX2-1/H1975 samples. Indeed, the open chromatin ATAC-seq reads tended to be enriched with a large number of genes at the transcription start sites (TSS) in shNKX2-1/H1975 samples in comparison with shCtrl/H1975, indicative of the potential role of NKX2-1 as a negative gene regulator of gene expression (FIG. 22A).

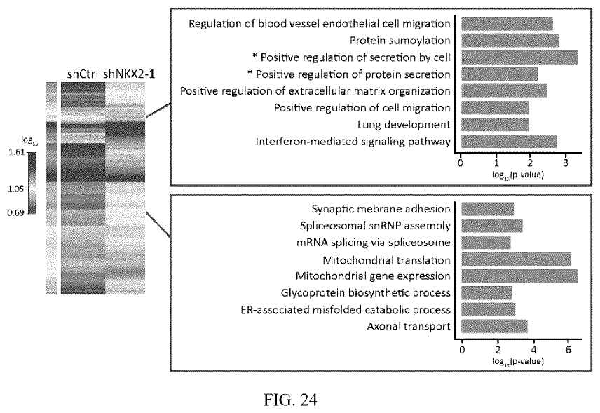

Generally, the distribution pattern of open chromatin peaks across different functional genomic elements exhibited an increase within less than 1 kb of the promoter region in shNKX2-1 sample (26.06%) when compared to the shCtrl/H1975 (16.32%) (FIG. 22B). To determine the changes in chromatin accessibility following NKX2-1 knockdown, we evaluated the differential accessibility between shNKX2-1/H1975 and shCtrl/H1975 cells across the genome. The enriched open chromatin regions resulting from NKX2-1 knockdown were predominantly located on chr18, chr13, chr6, and chr4 (FIG. 23). Interestingly, chr4 where the cluster of CXC chemokine genes is located, was one of the most chromatin-accessible chromosomes resultant from NKX2-1 knockdown. As was shown by GO-BP enrichment analysis, the enriched open chromatin regions resultant from NKX2-1 knockdown were mostly located at the promoters of genes associated with the positive regulation of secretion by cells and positive regulation of protein secretion (FIG. 24), which was consistent with our hypothesis. On the other hand, more closed chromatin structure in shNKX2-1/H1975 cells as compared to shCtrl/H1975 cells was present at the promoters of genes associated with mitochondrial gene expression and mitochondrial translation.

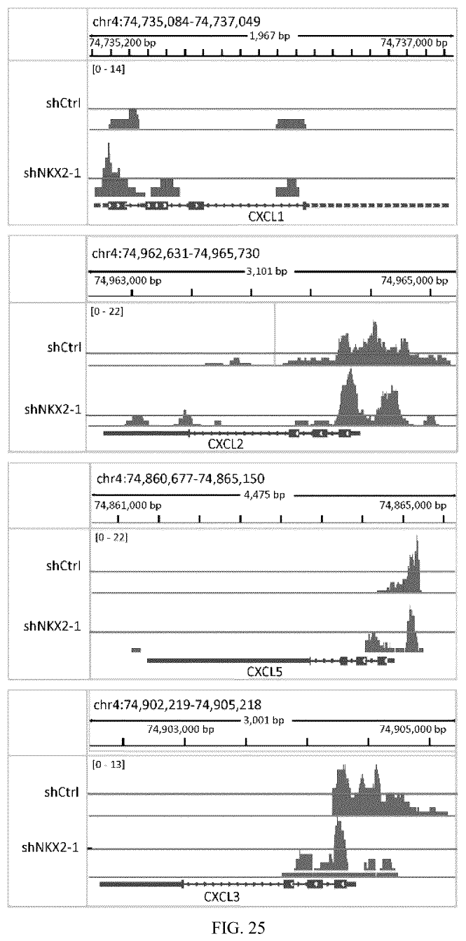

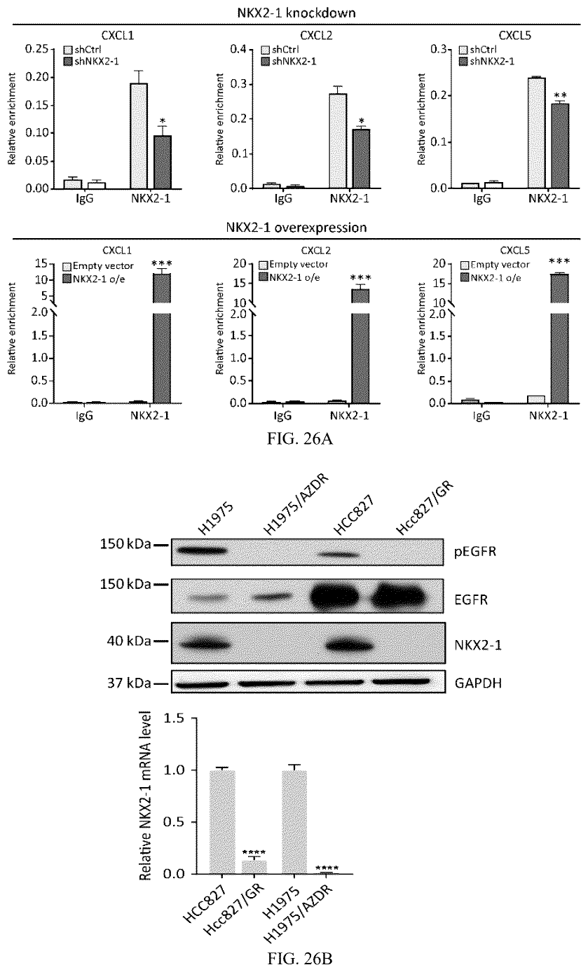

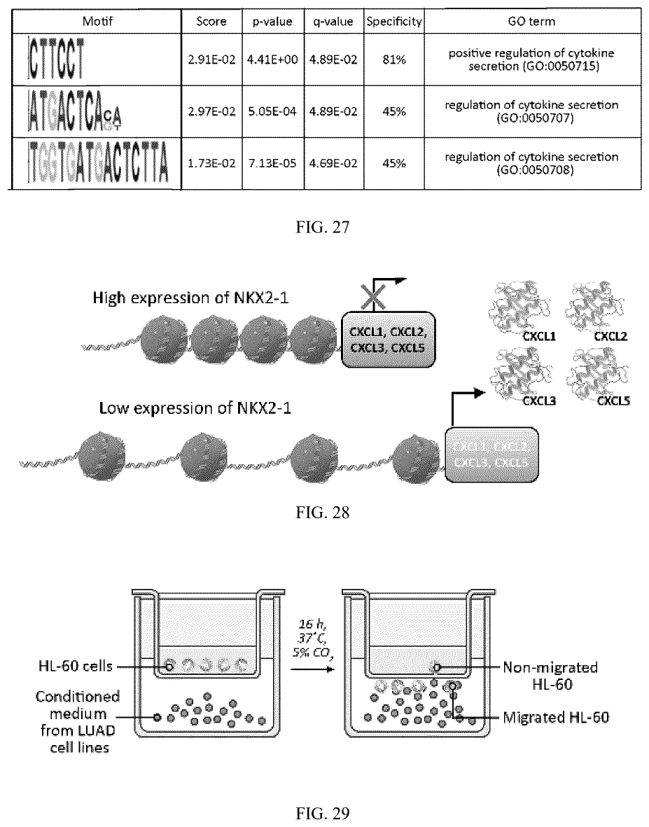

Notably, our ATAC-seq results showed an increase in open chromatin-associated reads at the promoter regions of CXCL1, CXCL2, and CXCL5 genes following the knockdown of NKX2-1 in H1975 cells (FIG. 25). This suggested that NKX2-1 could potentially modulate the chromatin structure at the promoter regions of these chemokine genes. We used ChIP-qPCR to validate this observation and showed that the knockdown of NKX2-1 in H1975 (NKX2-1-high cell line) led to a decrease in the occupancy of NKX2-1 at the promoter regions of CXCL1, CXCL2, and CXCL5 (FIG. 26A). In contrast, the overexpression of NKX2-1 in H1975/AZDR cells (NKX2-1-low cell line) as shown in FIG. 26B increased the occupancy of NKX2-1 on the promoter regions of the same genes. Next, we sought to identify the potential NKX2-1-binding sequence motifs that might be related to the regulation of cytokines expression. ATAC-seq reads were analyzed by using Find Individual Motif Occurrences (FIMO) software to predict NKX2-1-binding motif sequences. Our results showed three most prevalent NKX2-1-binding motif sequences in ATAC-seq reads close to the canonical motif sequence (FIG. 27). Collectively, our data suggest that the direct binding of NKX2-1 negatively regulates chromatin accessibility at the promoters of CXC chemokine genes (FIG. 28), which is consistent with the regulatory role of NKX2-1 on CXC chemokine expressions and secretions.

### Neutrophils Attracted by NKX2-1-Low Tumor Cells Exhibit Cancer-Promoting Properties

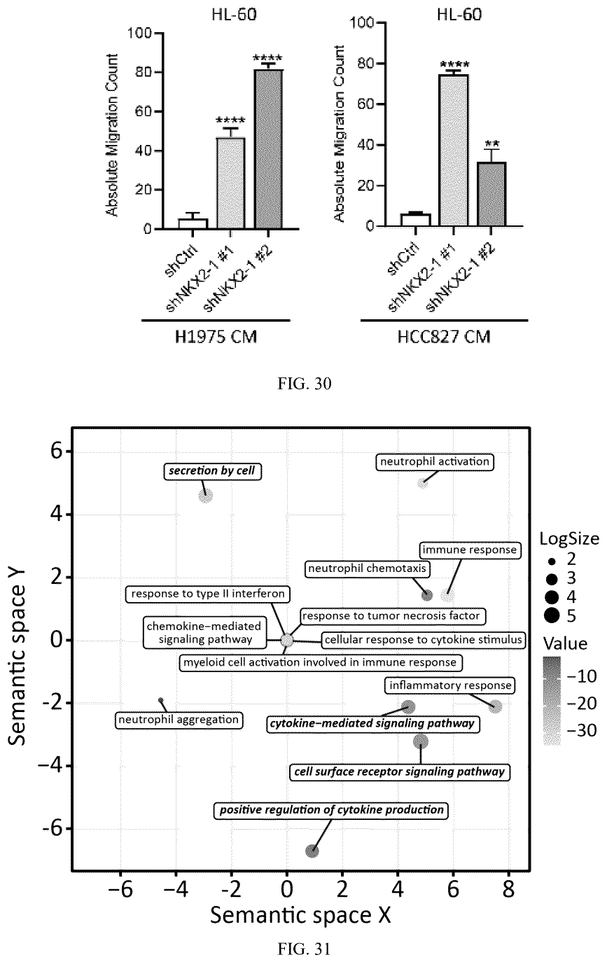

In light of the known role of CXC chemokines as potent neutrophil chemoattractants [32], we conducted a chemotaxis assay to validate the functionality of NKX2-1-suppressed CXC chemokines (FIG. 29). As was shown by transwell chemotaxis assay, HL-60 cell migration was significantly stimulated by the medium conditioned by shNKX2-1-transfected H1975 and HCC827 cells in contrast to the medium conditioned by shRNA-transfected control (shCtrl) (FIG. 30). To investigate the detailed global expression programs occurring in the recruited neutrophils, we performed RNA-seq analysis on HL-60 cells cultured in the medium conditioned by shCtrl and shNKX2-1-transfected H1975 cells. Revigo tool was utilized for GO analysis of the genes positively regulated in HL-60 cells upon culture with medium conditioned by shNKX2-1/H1975 cells. The results showed the enrichment of GO-BP terms such as secretion by cells, neutrophil activation, inflammatory response, immune response, neutrophil chemotaxis, etc. (FIG. 31).

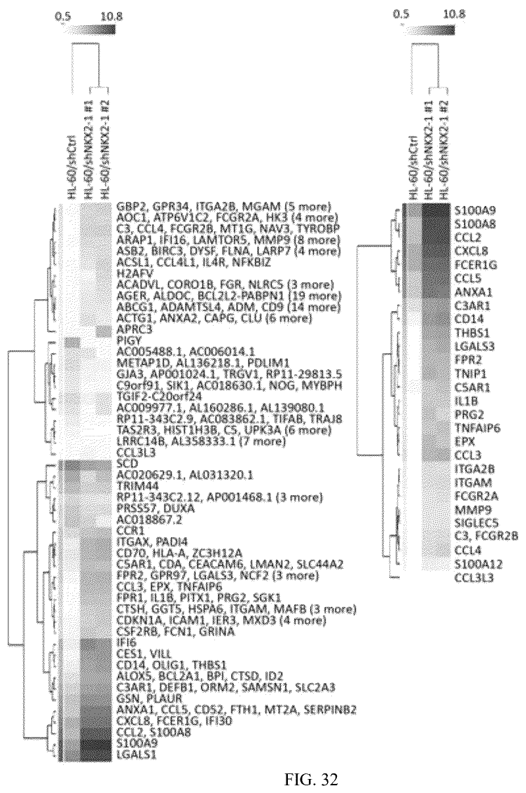

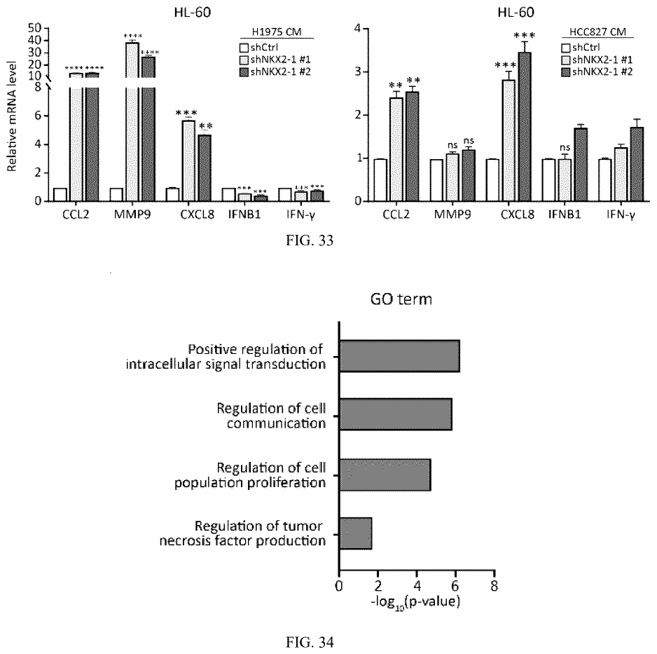

The phenotypic manifestation of neutrophils is related to their ability to adapt to different inflammatory contexts in the TME. For instance, previous reports have demonstrated that tumor-promoting or tumor-suppressing functions of neutrophils are determined by the functional characteristics exhibited through specific markers related to neutrophil activation and cytokine status [34]. To identify the neutrophil phenotypes, we applied hierarchical clustering to identify the distinct gene expression patterns in HL-60 cells cultured with the medium conditioned by NKX2-1 knockdown cells. The analysis showed an increase in the expression of specific pro-inflammatory genes such as CCL3, CCL5, ILIB, CXCL8, CCL4, among others (FIG. 32). Earlier studies have indicated that the antitumor phenotype (N1) of TANs is marked by the elevated levels of TNFa, CCL3, ICAM-1, and the decreased level of arginase. Conversely, the pro-tumor phenotype (N2) of TANs is characterized by high levels of expression of CCL2, CCL3, CCL4, CCL8, CCL12, CCL17, CXCL1, CXCL2, CXCL8, and CXCL16 chemokines [35]. To confirm the phenotypic effects on HL-60 cells cultured in the conditioned medium derived from shNKX2-1-transfected H1975 and HCC827 cells, we conducted qRT-PCR analysis, and our results revealed a significant increase in the mRNA expression levels of pro-tumor genes CCL2, MMP9, and CXCL8 (FIG. 33). Meanwhile, HL-60 treated by shNKX2-1/H1975-conditioned medium exhibited a significant decrease in the mRNA expression levels of antitumor genes encoding IFNß1 and IFN-γ. In contrast, shNKX2/HCC827 medium showed no significant impact on the expression levels of these genes (FIG. 6E).

Furthermore, Visium in situ capturing profiling was performed to identify and characterize the transcriptomes of the infiltrated neutrophils in in vivo mouse model. The GO-BP enrichment analysis of the genes overexpressed in the tumors across all regions derived from shNKX2-1/LL2 cells as compared to shCtrl/LL2 cells showed a prevalence of such processes as positive regulation of intracellular signal, regulation of cell communication, regulation of cell population proliferation, and regulation of tumor necrosis factor superfamily cytokine production (FIG. 34). By analyzing the regions within tumor cross sections with enriched neutrophil infiltrations, and we identified upregulation of neutrophil-related genes such as S100a9, Serpinel, Mt2, Nos2, Mt1, IL33, Adm, and Erol1 [36-38]. The regions with increased above-threshold expression of these neutrophil-related genes (−log 2 fold-change) were defined as infiltration-positive, while the regions with below-threshold expression were defined as infiltration-negative.

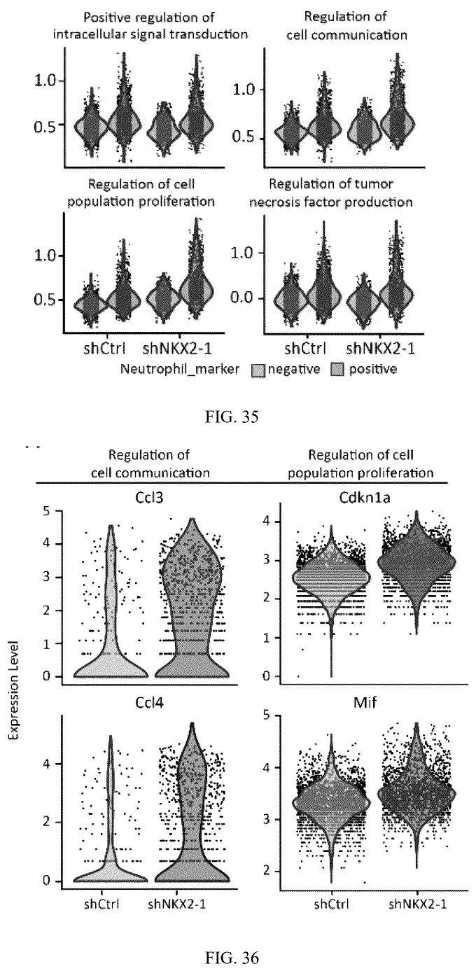

As shown in FIG. 35, our results revealed that neutrophil-positive regions exhibited the enrichment of GO-BP terms of cell communication and cell population proliferation compared to the neutrophil-negative regions in both shCtrl and shNKX2-1 tumors. At the same time, the neutrophil-negative regions exhibited a slight difference based on the GO-BP terms related to the regulation of cell communication and cell population proliferation. Our results also revealed that positive regulation of intracellular signal transduction and the regulation of tumor necrosis factor production were not affected in neutrophil-positive regions.

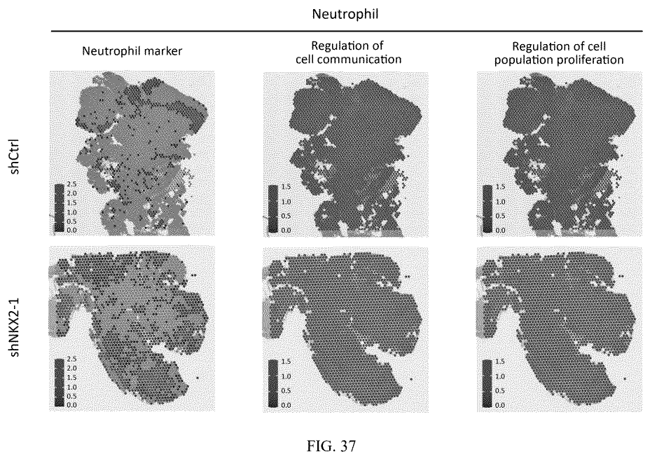

Ccl3 and Ccl4 were the most upregulated genes related to the regulation of cell communication in shNKX2-1 tumor-infiltrated neutrophils when compared to the shCtrl tumor-infiltrated neutrophils (FIG. 36), which was consistent with our initial observations. At the same time, Cdkn1a and Mif were the most upregulated genes implicated in the regulation of cell population proliferation in shNKX2-1 tumor-infiltrated neutrophils when compared to the shCtrl tumor-infiltrated neutrophils (FIG. 36). The abundance of neutrophil-positive regions was much higher in shNKX2-1 as compared to shCtrl tumors on the cross sections of Visium in situ capturing visualization, and these regions markedly overexpressed genes of regulation of cell communication and regulation of cell population proliferation GO-BP terms (FIG. 37). In summary, our findings suggest that low expression of NKX2-1 induces specific phenotypic properties in neutrophils, which potentially contribute to increased cancer progression through cell communication and cell proliferation. In other words, low levels of NKX2-1 in cancer cells may create a pro-tumor immune microenvironment, fostering malignant tumor development.

In Vivo Targeting of CXCLs/CXCR2 Signaling with SB225002 Reduces Tumor Growth and Neutrophil Infiltration

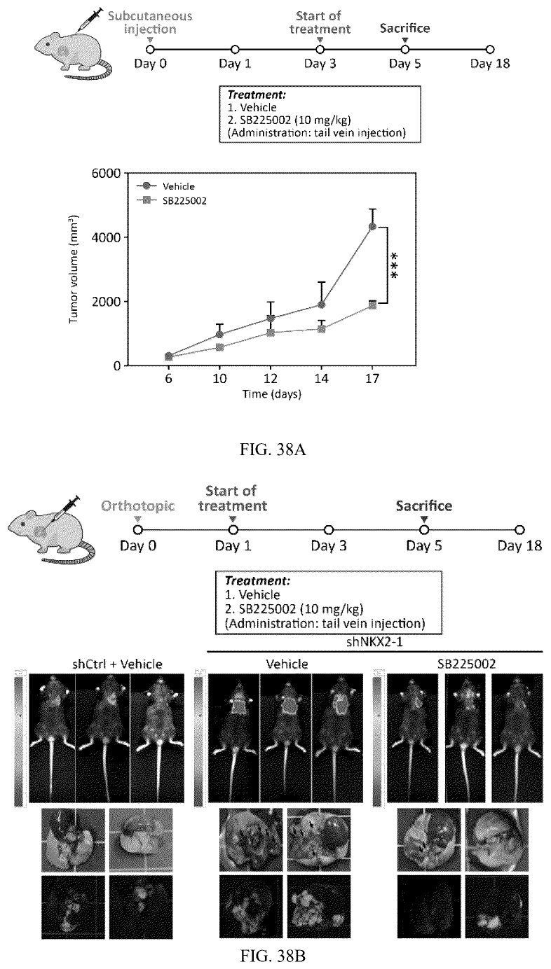

The identified NKX2-1-regulated CXC chemokines are known to share a common receptor, CXCR2. CXCR2 is a crucial chemokine receptor that facilitates neutrophil chemotaxis [39]. The CXCLs/CXCR2 signaling is associated with increased cancer progression in LUAD aside from its significant role in recruiting neutrophils to inflamed sites [14, 40, 41]. Previous reports have shown the potential of interfering with CXCLs/CXCR2 signaling to reduce tumor growth and enhance the efficiency of immunotherapy in different cancers [14, 40, 42]. Since our findings are indicative of the modulatory role of NKX2-1 in LUAD TME, we sought to investigate whether targeting the CXCL/CXCR2 signaling pathway could suppress NKX2-1-low LUAD tumor growth and neutrophil infiltration. shCtrl/LL2 and shNKX2-1/LL2 cells were orthotopically and subcutaneously injected with and without intravenous administration of SB225002. The experimental course for subcutaneous and orthotopic LUAD models was 18 days and 5 days, respectively, with SB225002 delivered every second day (FIGS. 38A and 38B). The gross necropsy findings showed a remarkable tumor suppression by CXCR2 antagonism compared with the vehicle control recipient mice upon tail vein administration of SB225002 in the subcutaneous mice model (FIG. 38A). Monitoring the tumor growth in the orthotopic injection model by using the IVIS imaging system also showed the suppression of tumor growth by CXCR2 antagonism (FIG. 38B). Altogether, our results demonstrate that low expression of NKX2-1 fosters tumor growth, and targeting CXCLs/CXCR2 axis with SB225002 mitigates the tumor growth induced by NKX2-1 downregulation.

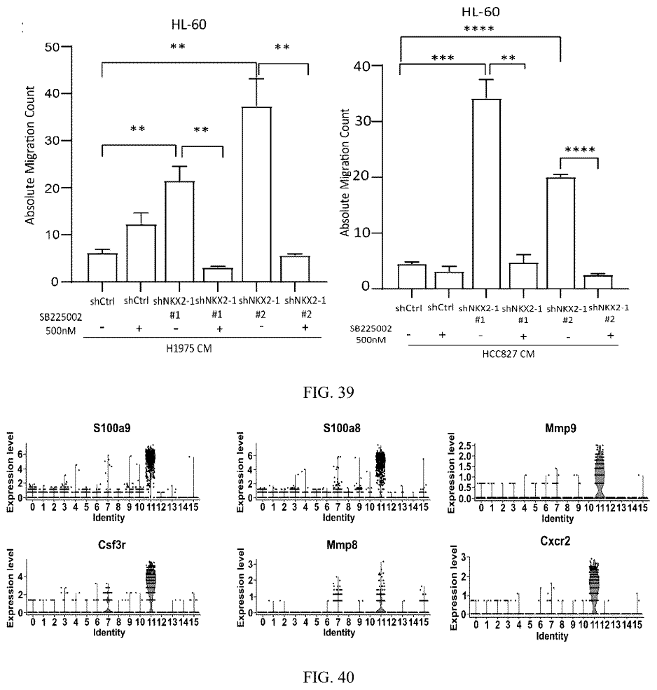

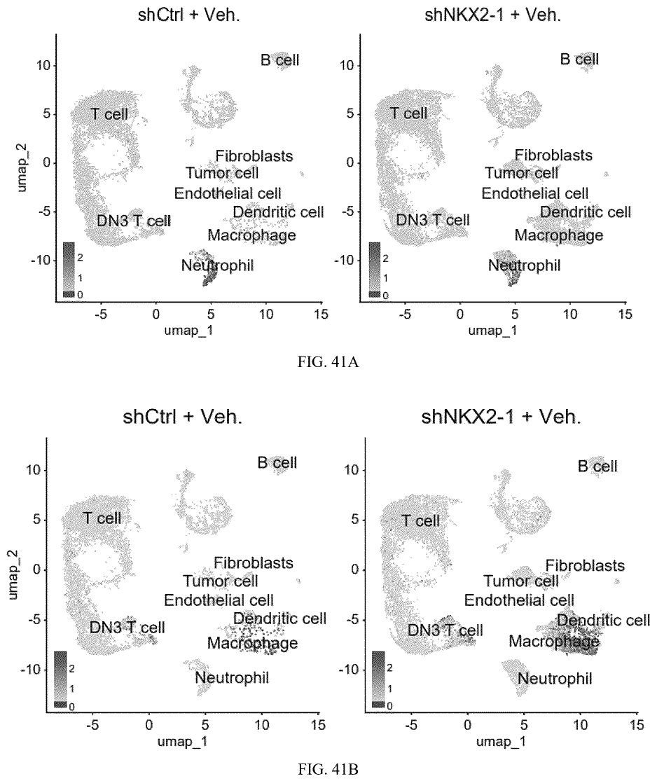

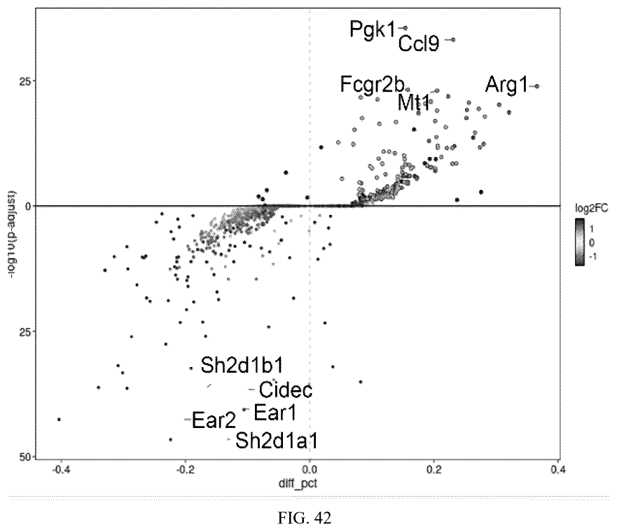

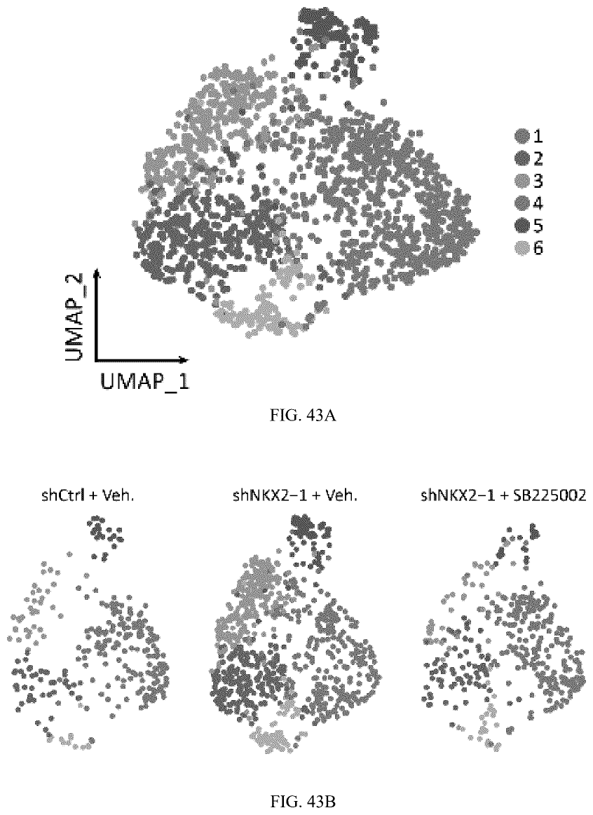

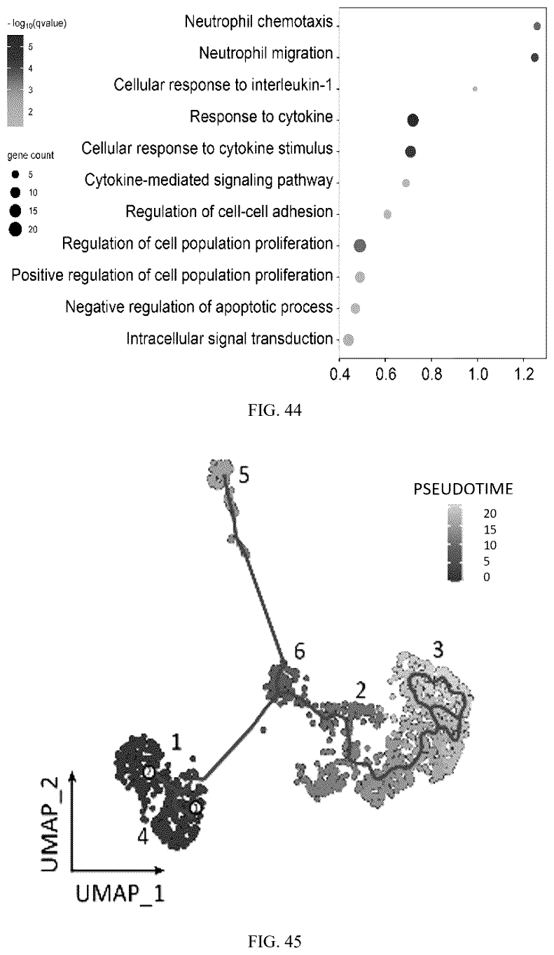

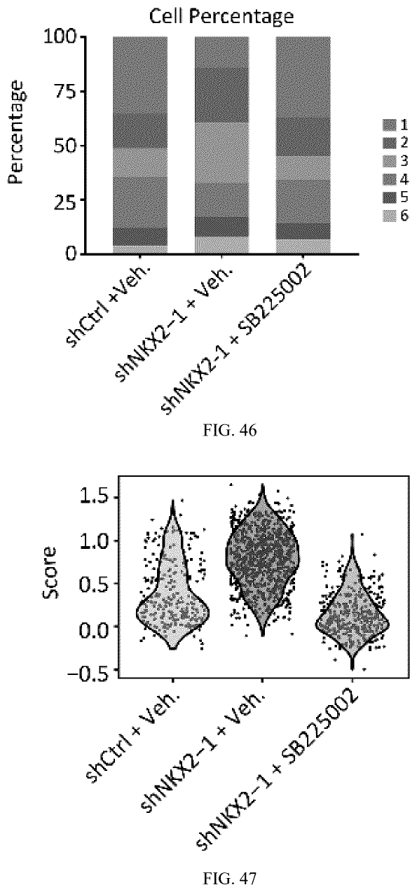

Next, we aimed to assess whether the CXCLs/CXCR2 signaling is essential for NKX2-1-dependent modulation of neutrophil recruitment and neutrophil infiltration. Chemotaxis assay showed that SB225002, a specific inhibitor of CXCR2, could effectively abrogate the migration of HL-60 cells stimulated by shNKX2-1/HCC827 and shNKX2-1/H1279 conditioned media (FIG. 39). scRNA-seq analysis was performed with a major focus on the neutrophil population after inhibiting CXCR2 with SB225002. Specifically, shCtrl/LL2 and shNKX2-1/LL2 cells were orthotopically implanted into mouse lungs with or without subsequent administration of SB225002 (FIG. 38B). The harvested tumors were dissociated into viable single cells. Different neutrophil markers were used to identify the neutrophil population using the FindAllMarkers package (FIG. 40), and UMAP analysis also indicated that only the neutrophil population expressed CXCR2 (FIG. 41A). In order to differentiate between neutrophils and myeloid-derived suppressor cells (MDSCs), given their similar phenotype and morphology [42], the expression of CCR2 was validated from our scRNA-seq analysis since it is a typical marker of MDSCs [43]. The result did not reveal any MDSC population; however, we found more CCR2-positive macrophages (FIG. 41B), and these macrophages were characterized by high expression of PgK1, Ccl9, Fcgr2b, Arg1, and Mt1 (FIG. 42). Unsupervised clustering was performed on the neutrophil population which resulted in six clusters (FIGS. 43A and 43B). Each subpopulation was independently validated. The GO-BP enrichment analysis of the genes overexpressed in the tumor cells derived from shNKX2-1/LL2 cells as compared to shCtrl/LL2 cells within cluster 3 showed a prevalence of neutrophil chemotaxis and migration, response to cytokine, regulation of cell population proliferation, among others (FIG. 44), which was similar to Visium in situ capturing profiling (FIG. 34). Indeed, NKX2-1 knockdown increased the neutrophil population, while SB225002 treatment decreased it. Additionally, pseudo-time analysis was used to delineate the single-cell lineage order based on the gene expression profile obtained from the scRNA-seq analysis (FIG. 45). The distribution pattern of cell percentages across six neutrophil clusters showed similarity between shCtrl/LL2-derived tumors with vehicle control and SB225002-treated shNKX2-1/LL2 tumor, but was different in shNKX2-1/LL2-derived tumor with vehicle control (FIG. 46). Moreover, unlike other clusters, cluster 3 was the most responsive to these experimental conditions, sharply increasing its proportion in shNKX2-1 tumors as compared to shCtrl, and conversely, demonstrating the most pronounced decreased in proportion in shNKX2-1 tumors treated with SB225002 (FIG. 46). This observation suggests that blocking the CXCLs/CXCR2 signaling can reverse the effect of NKX2-1 knockdown in terms of both the quantity and the landscape of infiltrated neutrophils. Notably, the neutrophil population of cluster 3 consistently exhibited an enrichment pattern across three different samples (shNKX2-1/LL2 tumors with vehicle administration, shCtrl/LL2 tumors with vehicle administration, and shNKX2-1/LL2 tumors with SB225002 administration). The result demonstrated significant enrichment in shNKX2-1+vehicle, compared to the shCtrl+vehicle and shNKX2-1+SB225002 (FIG. 46).

To gain deeper insights into the potential role of the infiltrated neutrophils in tumor progression, we explored the expression of the specific underlying cancer-promoting genes within cluster 3. As illustrated by a violin plot, significant regulation of cancer-promoting genes in shNKX2-1/LL2+vehicle compared to both shCtrl/LL2+vehicle and shNKX2-1/LL2+SB225002 (FIG. 7G). As shown in Table 4, we also explored the expression of the underlying cancer-promoting genes within cluster 3; these genes included Cdkn1a, Plaur, Ptgs2, Cox17, Lilrb4q, G0s2, Egr1, and Cxcl2, with previous reports suggesting their implication in increasing cancer progression.

TABLE 4
List of cancer-promoting genes from scRNA-seq analysis

| | | Gene | |
|---|---|---|---|
| No. | Gene name | Symbol | References |
| 1. | Cyclin Dependent Kinase Inhibitor 1A | CDKN1A | [1-3] |
| 2. | Plasminogen activator, urokinase | PLAUR | [4-6] |
| | receptor | | |
| 3. | Prostaglandin-Endoperoxide Synthase 2 | PTGS2 | [7-9] |
| 4. | Cytochrome C Oxidase Copper | COX17 | [10] |
| | Chaperone COX17 | | |
| 5. | Leukocyte Immunoglobulin Like | LILRB4 | [11-13] |
| | Receptor B4 | | |
| 6. | G0/G1 Switch 2 | G0S2 | [14, 15] |
| 7. | Early growth response protein 1 | EGR1 | [16-18] |
| 8. | C-X-C Motif Chemokine Ligand 2 | CXCL2 | [19, 20] |

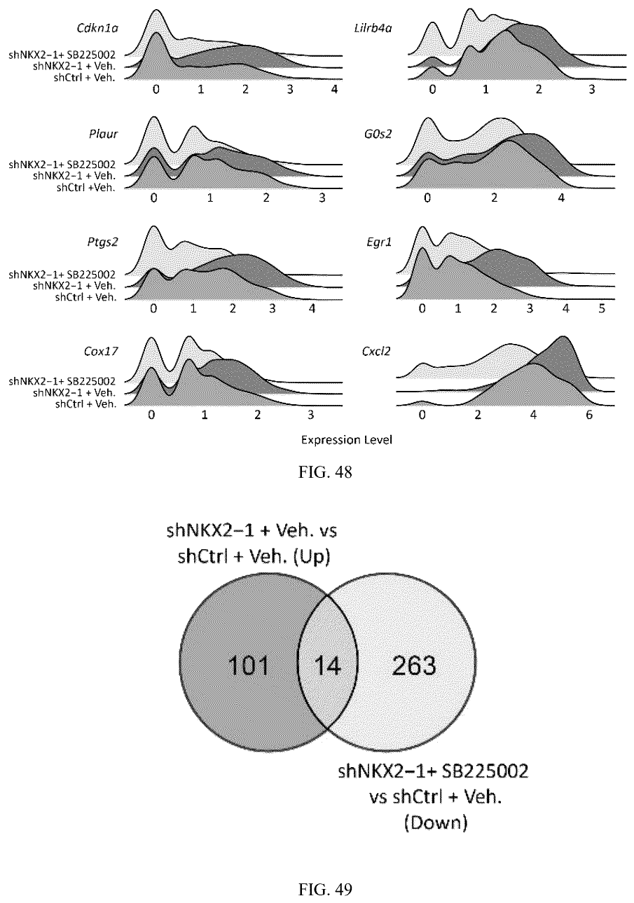

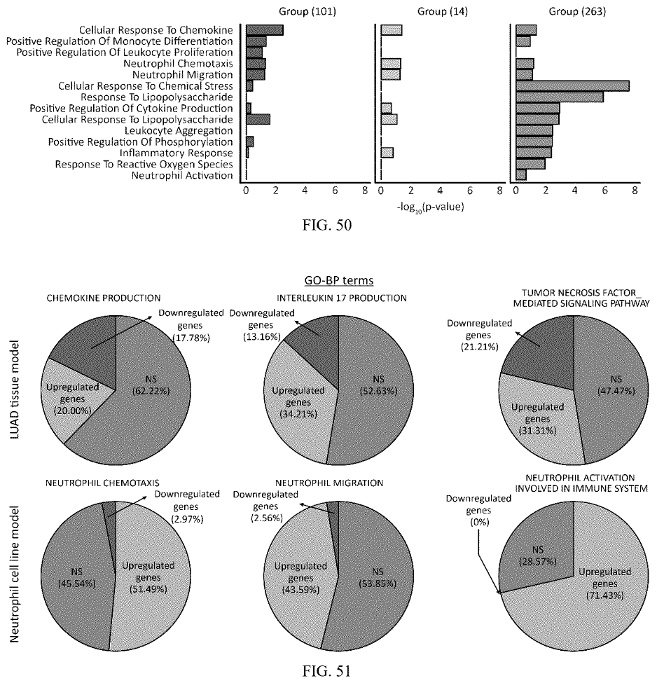

As illustrated by a ridgeline plot, the expression distribution of these genes was highly similar in shCtrl/LL2+vehicle and shNKX2-1/LL2+SB225002 samples, unlike in shNKX2-1/LL2+vehicle sample, where the expression was shifted to more upregulated mode (FIG. 48). To identify the genes regulated by NKX2-1 that may act in CXCLs/CXCR2 signaling-dependent manner, we compared the list of genes upregulated in shNKX2-1+vehicle compared to shCtrl+vehicle, i.e. genes under negative regulation by NKX2-1, with the list of genes downregulated genes in shNKX2-1+SB225002 compared to shNKX2-1+vehicle, i.e. the genes whose action can be attributed to CXCLs/CXCR2 and abrogation of chemokine stimulation. The result revealed that 115 genes were associated with NKX2-1 and cytokine stimulation, and 14 of those genes overlapped with the inhibition of CXCR2 signaling (FIG. 49). The 14 genes were Wfdc17, Bsg, Tnfrsf26, Plin2, Hilpda, Hspa5, Erol1, Egln3, Mif, Cxcl2, Bhlhe40, P4ha1, Nfkbiz and Chka. GO-BP analysis of these 14 genes indicated a decrease in the enrichment of cellular response to chemokine, neutrophil chemotaxis, neutrophil migration, positive regulation of cytokine production, cellular response to lipopolysaccharide, and inflammatory response as compared to the non-overlapping genes in shNKX2-1+vehicle vs shCtrl+vehicle and shNKX2-1+SB225002 vs shCtrl+vehicle (FIG. 50). Altogether, our scRNA-seq analysis indicated that the knockdown of NKX2-1 increased the neutrophil population and specifically attracted neutrophils with cancer-promoting properties. This phenomenon could be counteracted by inhibiting the CXCLs/CXCR2 signaling with CXCR2 antagonist. Our data demonstrated the pivotal role of the CXCLs/CXCR2 signaling in NKX2-1-low tumor progression and cancer-promoting neutrophil infiltration in LUAD. This suggests a potential method to control the malignant progression of NKX2-1-low LUAD tumors. Collectively, these findings emphasize that the CXCLs/CXCR2-dependent mechanism is essential for tumor progression and neutrophil infiltration in NKX2-1-low LUAD.

### Networks Engaged by NKX2-1-Low Tumor-Activated Neutrophils in In Vitro and In Vivo Models

In lung cancer, the activation of neutrophils in the TME introduces complexity to the inflamed environment by triggering additional mechanisms [26]. Our observations indicated that the downregulation of NKX2-1 promotes the recruitment and infiltration of neutrophils into LUAD tumors through the secretion of chemokines such as CXCL1, CXCL2, CXCL3, and CXCL5 into the TME, further fostering tumor-promoting effects. Notably, among the CXC chemokines, CXCL1, CXCL2, and CXCL5 are involved in the paracrine network that mediates tumor progression and metastasis [44]. To further evaluate the molecular events triggered by the tumor-promoting neutrophils attracted by NKX2-1-low LUAD tumors, we analyzed both scRNA-seq samples (NKX2-1-low tumors in comparison with the control) and RNA-seq data from HL-60 cells co-cultured with shNKX2-1/H1975 conditioned medium compared to HL-60 co-cultured with shCtrl/H1975 conditioned medium, i.e., in vivo and in vitro models, respectively.

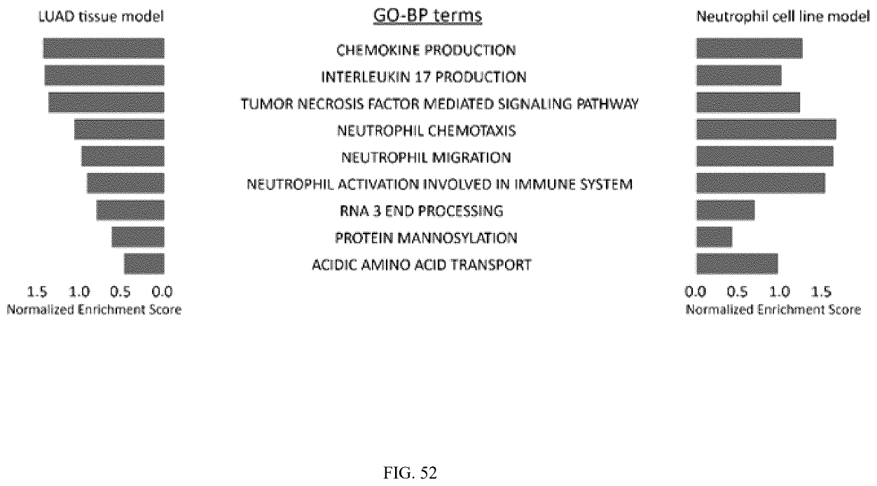

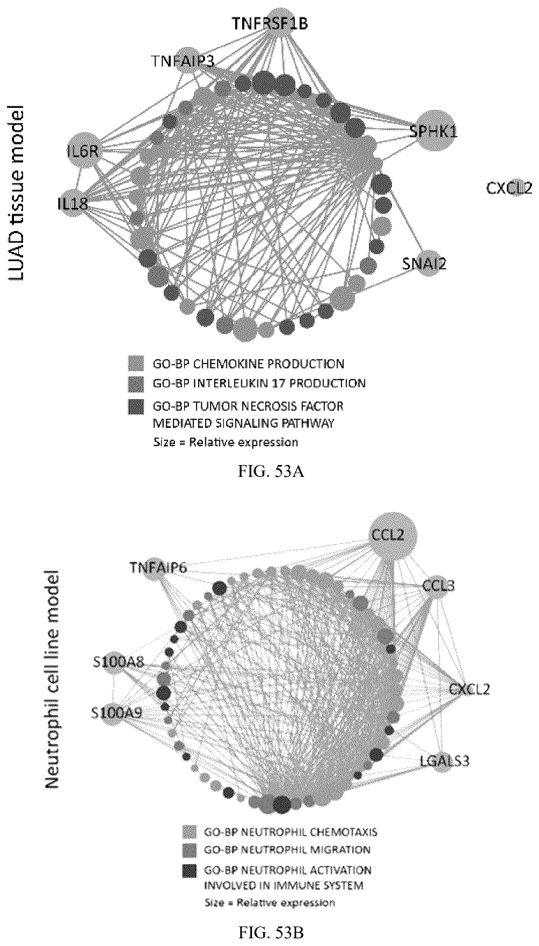

GO-BP enrichment analysis showed that neutrophils induced in NKX2-1-low LUAD tumor exhibited enrichment in chemokine production (20% upregulated genes), interleukin 17 (IL-17) production (34.21% upregulated genes), and tumor necrosis factor-mediated signaling pathway (31.31% upregulated genes) compared to the control tumor sample (FIG. 51). Previous report has shown that IL-17 production and tumor necrosis factor are related to tumor growth [44], this implies that the enrichment of tumor necrosis factor-mediated signaling pathway and IL-17 production in neutrophils underlies high inflammation leading to increased tumor growth. Additionally, HL-60 cells cultured in shNKX2-1/H1975-conditioned medium HL-60 showed enrichment in neutrophil chemotaxis (51.49% upregulated genes), neutrophil migration (43.59% upregulated genes), and neutrophil activation involved in the immune system (71.43% upregulated genes) GO-BP terms compared to the control group (FIG. 51). This suggested that neutrophils engaged by NKX2-1-low cancer cells play a role in promoting tumor progression by modulating the tumor surrounding with tumor-promoting molecules and inflammatory cells. Generally, both mouse in vivo and human in vitro neutrophil models exhibited enrichment of the same GO-BP terms, emphasizing the universality of the observed NKX2-1-dependent effects (FIG. 52). A network analysis was conducted to further validate the genes involved in these enriched pathways. The results indicated a significant gene communication between Tnfrsf1b, Tnfaip3, Sphk1, Snai2, Il6r, and Il18 in NKX2-1-low tumors (FIG. 53A) while in HL-60 exposed to shNKX2-1/H1975 conditioned medium, a significant gene communication between CXCL2, CCL2, CCL3, LGALS3, TNFAIP6, S100A8 and S100A9 was inferred (FIG. 53B).

Altogether, our studies demonstrated the modulatory role of NKX2-1 in the immune microenvironment of LUAD through the regulation of chemokine expression and secretion. This promotes recruitment and infiltration of tumor-promoting neutrophils into the tumor, further contributing to tumor progression.

In the present invention, we demonstrated that NKX2-1 can suppress the expression of CXC chemokines and trigger IL-17 production, leading to an increase in neutrophil recruitment. Previous reports have established that NKX2-1 can directly bind to the proximal promoter regions or the various intragenic and intergenic regions of the regulated genes [56]. On account of this observation, our ATAC-seq analysis revealed that the downregulation of NKX2-1 resulted in open chromatin structure at the promoter-TSS of CXCL1, CXCL2, and CXCL5 genes. Furthermore, we identified NKX2-1-binding motifs that could govern the regulation of these cytokine genes. This implies that the binding activity of NKX2-1 at the regulatory elements of CXCL1, CXCL2, and CXCL5 promotes the repressive states of these genes. Notably, this is consistent with prior studies showing NKX2-1 binding at the promoter and the first exon of murine Cxcl5 [19]. Consequently, targeting the CXCLs/CXCR2 axis could potentially enhance the clinical outcomes for NKX2-1-low LUAD patients.

To characterize the neutrophil population influenced by NKX2-1 loss in LUAD malignancy, we applied scRNA-seq and Visium in situ capturing to investigate the immune microenvironment heterogeneity in NKX2-1-low tumors. These novel tools offer unprecedented insight into cellular biology [57, 58]. Our quantitative analyses revealed that NKX2-1 downregulation led to increased neutrophil infiltration, a phenomenon mediated by CXC chemokines. Notably, this effect was abrogated by CXCR2 antagonism. While most studies often focus on neutrophil plasticity, cellular density, or maturation, the full potential of the genes expressed by neutrophils in the TME remains underexplored. It is crucial to highlight that our scRNA-seq and Visium in situ capturing unveiled the expression of cancer-promoting genes in the infiltrated neutrophils. This identification was achieved through unsupervised clustering, which revealed the broad gene expression patterns within the heterogenous neutrophil population. Notably, one of such clusters exhibited a particularly high pattern of expression of cancer-promoting genes, which included Cdkn1a, Plaur, Ptgs2, Cox17, Lilrb4q, G0s2, Egr1, and Cxcl2, many of them encoding secreted tumor-promoting factors. Neutrophil infiltration correlates with tumor aggressiveness via tumor grades in human gliomas [27], and previous reports have also shown that the infiltration of neutrophils is related to an aggressive type of pancreatic tumor [59]. Importantly, the present invention demonstrates that the interplay between NKX2-1, CXC chemokine axis, and neutrophil infiltration is associated with tumor aggressiveness marked by high expression of tumor-promoting genes by infiltrated neutrophils.

In summary, it is delineated that the role of NKX2-1 as a bona fide modulator of the immune tumor microenvironment in NKX2-1-low LUAD, whose loss induces the expression/secretion of CXC chemokines leading to increased recruitment and infiltration of tumor-promoting neutrophils. These neutrophils exhibit cancer-promoting properties with strong cell-cell communication within tumor tissue. This creates a pro-oncogenic TME, which ultimately drives increased tumor progression. Our findings provided compelling evidence for the involvement of the NKX2-1/CXC chemokine signaling axis in mediating neutrophil infiltration and LUAD progression. Targeting the NKX2-1/CXC chemokine signaling may represent a strategy of treatment to suppress tumorigenesis and improve the survival outcome of LUAD patients with NKX2-1-low malignant tumors.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment. Conversely, various features that are described in the context of a single embodiment or example can also be implemented in multiple embodiments or examples separately or in any appropriate suitable sub-combination.

### REFERENCES

1. Fang Z, Han X, Chen Y, Tong X, Xue Y, Yao S, et al. Oxidative stress-triggered Wnt signaling perturbation characterizes the tipping point of lung adeno-to-squamous transdifferentiation. Signal Transduct Target Ther. 2023; 8(1):16. Epub 20230111. doi: 10.1038/s41392-022-01227-0. PubMed PMID: 36627278; PubMed Central PMCID: PMCPMC9832009.
2. Yu X, Dong Z, Wang W, Mao S, Pan Y, Liu Y, et al. Adenocarcinoma of High-Grade Patterns Associated with Distinct Outcome of First-Line Chemotherapy or EGFR-TKIs in Patients of Relapsed Lung Cancer. Cancer Manag Res. 2021; 13:3981-90. Epub 20210517. doi: 10.2147/cmar.S302545. PubMed PMID: 34040439; PubMed Central PMCID: PMCPMC8139732.
3. Altorki N K, Markowitz G J, Gao D, Port J L, Saxena A, Stiles B, et al. The lung microenvironment: an important regulator of tumour growth and metastasis. Nat Rev Cancer. 2019; 19(1):9-31. doi: 10.1038/s41568-018-0081-9. PubMed PMID: 30532012; PubMed Central PMCID: PMCPMC6749995.
4. Saito R A, Watabe T, Horiguchi K, Kohyama T, Saitoh M, Nagase T, et al. Thyroid transcription factor-1 inhibits transforming growth factor-beta-mediated epithelial-to-mesenchymal transition in lung adenocarcinoma cells. Cancer Res. 2009; 69(7):2783-91. Epub 20090317. doi: 10.1158/0008-5472.Can-08-3490. PubMed PMID: 19293183.
5. Chen S, Tang J, Liu F, Li W, Yan T, Shangguan D, et al. Changes of tumor microenvironment in non-small cell lung cancer after TKI treatments. Front Immunol. 2023; 14:1094764. Epub 20230306. doi: 10.3389/fimmu.2023.1094764. PubMed PMID: 36949948; PubMed Central PMCID: PMCPMC10025329.
6. Maffuid K, Cao Y. Decoding the Complexity of Immune-Cancer Cell Interactions: Empowering the Future of Cancer Immunotherapy. Cancers (Basel). 2023; 15(16). Epub 20230821. doi: 10.3390/cancers15164188. PubMed PMID: 37627216; PubMed Central PMCID: PMCPMC10453128.
7. Rosales C, Demaurex N, Lowell C A, Uribe-Querol E. Neutrophils: Their Role in Innate and Adaptive Immunity. J Immunol Res. 2016; 2016:1469780. Epub 20160224. doi: 10.1155/2016/1469780. PubMed PMID: 27006954; PubMed Central PMCID: PMCPMC4783580.
8. Quail D F, Amulic B, Aziz M, Barnes B J, Eruslanov E, Fridlender Z G, et al. Neutrophil phenotypes and functions in cancer: A consensus statement. J Exp Med. 2022; 219(6). Epub 20220506. doi: 10.1084/jem.20220011. PubMed PMID: 35522219; PubMed Central PMCID: PMCPMC9086501.
9. Ley K, Hoffman H M, Kubes P, Cassatella M A, Zychlinsky A, Hedrick C C, et al. Neutrophils: New insights and open questions. Sci Immunol. 2018; 3(30). Epub 20181207. doi: 10.1126/sciimmunol.aat4579. PubMed PMID: 30530726.
10. Eash K J, Greenbaum A M, Gopalan P K, Link D C. CXCR2 and CXCR4 antagonistically regulate neutrophil trafficking from murine bone marrow. J Clin Invest. 2010; 120(7):2423-31. doi: 10.1172/jci41649. PubMed PMID: 20516641; PubMed Central PMCID: PMCPMC2898597.
11. Coffelt S B, Wellenstein M D, de Visser K E. Neutrophils in cancer: neutral no more. Nat Rev Cancer. 2016; 16(7):431-46. Epub 20160610. doi: 10.1038/nrc.2016.52. PubMed PMID: 27282249.
12. Bonecchi R, Mantovani A, Jaillon S. Chemokines as Regulators of Neutrophils: Focus on Tumors, Therapeutic Targeting, and Immunotherapy. Cancers (Basel). 2022; 14(3). Epub 20220128. doi: 10.3390/cancers14030680. PubMed PMID: 35158948; PubMed Central PMCID: PMCPMC8833344.
13. Alfaro C, Teijeira A, Oñate C, Pérez G, Sanmamed M F, Andueza M P, et al. Tumor-Produced Interleukin-8 Attracts Human Myeloid-Derived Suppressor Cells and Elicits Extrusion of Neutrophil Extracellular Traps (NETs). Clin Cancer Res. 2016; 22(15):3924-36. Epub 20160308. doi: 10.1158/1078-0432.Ccr-15-2463. PubMed PMID: 26957562.
14. Cheng Y, Mo F, Li Q, Han X, Shi H, Chen S, et al. Targeting CXCR2 inhibits the progression of lung cancer and promotes therapeutic effect of cisplatin. Mol Cancer. 2021; 20(1):62. Epub 20210404. doi: 10.1186/s12943-021-01355-1. PubMed PMID: 33814009; PubMed Central PMCID: PMCPMC8019513.
15. Unver N. Identification of the dominant angiogenic CXCL class chemokines associated with non-small cell lung cancer via bioinformatics tools. Med Oncol. 2021; 38(6):68. Epub 20210513. doi: 10.1007/s12032-021-01517-7. PubMed PMID: 33983509.
16. Tang S, Xue Y, Qin Z, Fang Z, Sun Y, Yuan C, et al. Counteracting lineage-specific transcription factor network finely tunes lung adeno-to-squamous transdifferentiation through remodeling tumor immune microenvironment. National Science Review. 2023; 10(4). doi: 10.1093/nsr/nwad028.
17. Yang L, Lin M, Ruan W J, Dong L L, Chen E G, Wu X H, et al. Nkx2-1: a novel tumor biomarker of lung cancer. J Zhejiang Univ Sci B. 2012; 13(11):855-66. doi: 10.1631/jzus.B1100382. PubMed PMID: 23125078; PubMed Central PMCID: PMCPMC3494024.
18. Mollaoglu G, Jones A, Wait S J, Mukhopadhyay A, Jeong S, Arya R, et al. The Lineage-Defining Transcription Factors SOX2 and NKX2-1 Determine Lung Cancer Cell Fate and Shape the Tumor Immune Microenvironment. Immunity. 2018; 49(4):764-79.e9. doi: 10.1016/j.immuni.2018.09.020. PubMed PMID: 30332632; PubMed Central PMCID: PMCPMC6197489.
19. Snyder E L, Watanabe H, Magendantz M, Hoersch S, Chen T A, Wang D G, et al. Nkx2-1 represses a latent gastric differentiation program in lung adenocarcinoma. Mol Cell. 2013; 50(2):185-99. Epub 20130321. doi: 10.1016/j.molcel.2013.02.018. PubMed PMID: 23523371; PubMed Central PMCID: PMCPMC3721642.
20. Ferone G, Song J Y, Sutherland K D, Bhaskaran R, Monkhorst K, Lambooij J P, et al. SOX2 Is the Determining Oncogenic Switch in Promoting Lung Squamous Cell Carcinoma from Different Cells of Origin. Cancer Cell. 2016; 30(4):519-32. doi: 10.1016/j.ccell.2016.09.001. PubMed PMID: 27728803; PubMed Central PMCID: PMCPMC5065004.
21. Gontan C, de Munck A, Vermeij M, Grosveld F, Tibboel D, Rottier R. Sox2 is important for two crucial processes in lung development: branching morphogenesis and epithelial cell differentiation. Dev Biol. 2008; 317(1):296-309. Epub 20080229. doi: 10.1016/j.ydbio.2008.02.035. PubMed PMID: 18374910.
22. Winslow M M, Dayton T L, Verhaak R G, Kim-Kiselak C, Snyder E L, Feldser D M, et al. Suppression of lung adenocarcinoma progression by Nkx2-1. Nature. 2011; 473(7345):101-4. Epub 20110406. doi: 10.1038/nature09881. PubMed PMID: 21471965; PubMed Central PMCID: PMCPMC3088778.
23. Fridman W H, Zitvogel L, Sautès-Fridman C, Kroemer G. The immune contexture in cancer prognosis and treatment. Nat Rev Clin Oncol. 2017; 14(12):717-34. Epub 20170725. doi: 10.1038/nrclinonc.2017.101. PubMed PMID: 28741618.
24. Wu L, Zhang X H. Tumor-Associated Neutrophils and Macrophages-Heterogenous but Not Chaotic. Front Immunol. 2020; 11:553967. Epub 20201202. doi: 10.3389/fimmu.2020.553967. PubMed PMID: 33343560; PubMed Central PMCID: PMCPMC7738476.
25. Li T, Fan J, Wang B, Traugh N, Chen Q, Liu J S, et al. TIMER: A Web Server for Comprehensive Analysis of Tumor-Infiltrating Immune Cells. Cancer Res. 2017; 77(21):e108-e10. doi: 10.1158/0008-5472.Can-17-0307. PubMed PMID: 29092952; PubMed Central PMCID: PMCPMC6042652.
26. Zhou J, Liu H, Jiang S, Wang W. Role of tumor-associated neutrophils in lung cancer (Review). Oncol Lett. 2023; 25(1):2. Epub 20221108. doi: 10.3892/ol.2022.13588. PubMed PMID: 36419755; PubMed Central PMCID: PMCPMC9677518.
27. Fossati G, Ricevuti G, Edwards S W, Walker C, Dalton A, Rossi M L. Neutrophil infiltration into human gliomas. Acta Neuropathol. 1999; 98(4):349-54. doi: 10.1007/s004010051093. PubMed PMID: 10502039.
28. Wang Q, Zhi Y, Zi M, Mo Y, Wang Y, Liao Q, et al. Spatially Resolved Transcriptomics Technology Facilitates Cancer Research. Adv Sci (Weinh). 2023; 10(30):e2302558. Epub 2023 Aug. 27. doi: 10.1002/advs.202302558. PubMed PMID: 37632718; PubMed Central PMCID: PMCPMC10602551.
29. Cambier S, Gouwy M, Proost P. The chemokines CXCL8 and CXCL12: molecular and functional properties, role in disease and efforts towards pharmacological intervention. Cell Mol Immunol. 2023; 20(3):217-51. Epub 20230201. doi: 10.1038/s41423-023-00974-6. PubMed PMID: 36725964; PubMed Central PMCID: PMCPMC9890491.
30. Hamdan H, Liu H, Li C, Jones C, Lee M, deLemos R, et al. Structure of the human Nkx2.1 gene. Biochimica et Biophysica Acta (BBA)-Gene Structure and Expression. 1998; 1396(3):336-48. doi: https://doi.org/10.1016/S0167-4781(97) 00210-8.
31. Buenrostro J D, Wu B, Chang H Y, Greenleaf W J. ATAC-seq: A Method for Assaying Chromatin Accessibility Genome-Wide. Curr Protoc Mol Biol. 2015; 109:21.9.1-. 9.9. Epub 20150105. doi: 10.1002/0471142727.mb2129s109. PubMed PMID: 25559105; PubMed Central PMCID: PMCPMC4374986.
32. Metzemaekers M, Gouwy M, Proost P. Neutrophil chemoattractant receptors in health and disease: double-edged swords. Cell Mol Immunol. 2020; 17(5):433-50. Epub 20200401. doi: 10.1038/s41423-020-0412-0. PubMed PMID: 32238918; PubMed Central PMCID: PMCPMC7192912.
33. Giese M A, Hind L E, Huttenlocher A. Neutrophil plasticity in the tumor microenvironment. Blood. 2019; 133(20):2159-67. Epub 20190321. doi: 10.1182/blood-2018-11-844548. PubMed PMID: 30898857; PubMed Central PMCID: PMCPMC6524564.
34. Masucci M T, Minopoli M, Carriero M V. Tumor Associated Neutrophils. Their Role in Tumorigenesis, Metastasis, Prognosis and Therapy. Front Oncol. 2019; 9:1146. Epub 20191115. doi: 10.3389/fonc.2019.01146. PubMed PMID: 31799175; PubMed Central PMCID: PMCPMC6874146.
35. Fridlender Z G, Albelda S M. Tumor-associated neutrophils: friend or foe? Carcinogenesis. 2012; 33(5):949-55. Epub 20120316. doi: 10.1093/carcin/bgs123. PubMed PMID: 22425643.
36. Zilionis R, Engblom C, Pfirschke C, Savova V, Zemmour D, Saatcioglu H D, et al. Single-Cell Transcriptomics of Human and Mouse Lung Cancers Reveals Conserved Myeloid Populations across Individuals and Species. Immunity. 2019; 50(5):1317-34.e10. Epub 20190409. doi: 10.1016/j.immuni.2019.03.009. PubMed PMID: 30979687; PubMed Central PMCID: PMCPMC6620049.
37. Tsukahara Y, Morisaki T, Kojima M, Uchiyama A, Tanaka M. iNOS expression by activated neutrophils from patients with sepsis. ANZ J Surg. 2001; 71(1):15-20. doi: 10.1046/j.1440-1622.2001.02025.x. PubMed PMID: 11167591.
38. Subramanian Vignesh K, Deepe G S, Jr. Metallothioneins: Emerging Modulators in Immunity and Infection. Int J Mol Sci. 2017; 18(10). Epub 20171023. doi: 10.3390/ijms18102197. PubMed PMID: 29065550; PubMed Central PMCID: PMCPMC5666878.
39. Jamieson T, Clarke M, Steele C W, Samuel M S, Neumann J, Jung A, et al. Inhibition of CXCR2 profoundly suppresses inflammation-driven and spontaneous tumorigenesis. J Clin Invest. 2012; 122(9):3127-44. Epub 20120827. doi: 10.1172/jci61067. PubMed PMID: 22922255; PubMed Central PMCID: PMCPMC3428079.
40. Cheng Y, Ma X L, Wei Y Q, Wei X W. Potential roles and targeted therapy of the CXCLs/CXCR2 axis in cancer and inflammatory diseases. Biochim Biophys Acta Rev Cancer. 2019; 1871(2):289-312. Epub 20190129. doi: 10.1016/j.bbcan.2019.01.005. PubMed PMID: 30703432.
41. Nywening™, Belt B A, Cullinan D R, Panni R Z, Han B J, Sanford D E, et al. Targeting both tumour-associated CXCR2 (+) neutrophils and CCR2 (+) macrophages disrupts myeloid recruitment and improves chemotherapeutic responses in pancreatic ductal adenocarcinoma. Gut. 2018; 67(6):1112-23. Epub 20171201. doi: 10.1136/gutjnl-2017-313738. PubMed PMID: 29196437; PubMed Central PMCID: PMCPMC5969359.
42. Zhang W, Wang H, Sun M, Deng X, Wu X, Ma Y, et al. CXCL5/CXCR2 axis in tumor microenvironment as potential diagnostic biomarker and therapeutic target. Cancer Commun (Lond). 2020; 40(2-3):69-80. doi: 10.1002/cac2.12010. PubMed PMID: 32237072; PubMed Central PMCID: PMCPMC7163794.
43. Lesokhin A M, Hohl™, Kitano S, Cortez C, Hirschhorn-Cymerman D, Avogadri F, et al. Monocytic CCR2 (+) myeloid-derived suppressor cells promote immune escape by limiting activated CD8 T-cell infiltration into the tumor microenvironment. Cancer Res. 2012; 72(4):876-86. Epub 20111215. doi: 10.1158/0008-5472.Can-11-1792. PubMed PMID: 22174368; PubMed Central PMCID: PMCPMC3288305.
44. Sionov R V, Fridlender Z G, Granot Z. The Multifaceted Roles Neutrophils Play in the Tumor Microenvironment. Cancer Microenvironment. 2015; 8(3):125-58. doi: 10.1007/s12307-014-0147-5.
45. Yamaguchi T, Hosono Y, Yanagisawa K, Takahashi T. NKX2-1/TTF-1: an enigmatic oncogene that functions as a double-edged sword for cancer cell survival and progression. Cancer Cell. 2013; 23(6):718-23. doi: 10.1016/j.ccr.2013.04.002. PubMed PMID: 23763999.
46. Moisés J, Navarro A, Santasusagna S, Viñolas N, Molins L, Ramirez J, et al. NKX2-1 expression as a prognostic marker in early-stage non-small-cell lung cancer. BMC Pulm Med. 2017; 17(1):197. Epub 20171213. doi: 10.1186/s12890-017-0542-z. PubMed PMID: 29237428; PubMed Central PMCID: PMCPMC5727907.
47. Kendall J, Liu Q, Bakleh A, Krasnitz A, Nguyen K C, Lakshmi B, et al. Oncogenic cooperation and coamplification of developmental transcription factor genes in lung cancer. Proc Natl Acad Sci USA. 2007; 104(42):16663-8. Epub 20071009. doi: 10.1073/pnas.0708286104. PubMed PMID: 17925434; PubMed Central PMCID: PMCPMC2034240.
48. Tanaka H, Yanagisawa K, Shinjo K, Taguchi A, Maeno K, Tomida S, et al. Lineage-specific dependency of lung adenocarcinomas on the lung development regulator TTF-1. Cancer Res. 2007; 67(13):6007-11. doi: 10.1158/0008-5472.Can-06-4774. PubMed PMID: 17616654.
49. Kwei K A, Kim Y H, Girard L, Kao J, Pacyna-Gengelbach M, Salari K, et al. Genomic profiling identifies TITF1 as a lineage-specific oncogene amplified in lung cancer. Oncogene. 2008; 27(25):3635-40. Epub 20080121. doi: 10.1038/sj.one.1211012. PubMed PMID: 18212743; PubMed Central PMCID: PMCPMC2903002.
50. Hou S, Han X, Ji H. Squamous Transition of Lung Adenocarcinoma and Drug Resistance. Trends Cancer. 2016; 2(9):463-6. Epub 20160902. doi: 10.1016/j.trecan.2016.08.002. PubMed PMID: 28741476.
51. Schoenfeld A J, Chan J M, Kubota D, Sato H, Rizvi H, Daneshbod Y, et al. Tumor Analyses Reveal Squamous Transformation and Off-Target Alterations As Early Resistance Mechanisms to First-line Osimertinib in EGFR-Mutant Lung Cancer. Clin Cancer Res. 2020; 26(11):2654-63. Epub 20200107. doi: 10.1158/1078-0432.Ccr-19-3563. PubMed PMID: 31911548; PubMed Central PMCID: PMCPMC7448565.
52. Levin P A, Mayer M, Hoskin S, Sailors J, Oliver D H, Gerber D E. Histologic Transformation from Adenocarcinoma to Squamous Cell Carcinoma as a Mechanism of Resistance to EGFR Inhibition. J Thorac Oncol. 2015; 10(9):e86-e8. doi: 10.1097/jto.0000000000000571. PubMed PMID: 26291017; PubMed Central PMCID: PMCPMC5158309.
53. Saintigny P, Massarelli E, Lin S, Ahn Y H, Chen Y, Goswami S, et al. CXCR2 expression in tumor cells is a poor prognostic factor and promotes invasion and metastasis in lung adenocarcinoma. Cancer Res. 2013; 73(2):571-82. Epub 20121130. doi: 10.1158/0008-5472.Can-12-0263. PubMed PMID: 23204236; PubMed Central PMCID: PMCPMC3548940.
54. Akbay E A, Koyama S, Liu Y, Dries R, Bufe L E, Silkes M, et al. Interleukin-17A Promotes Lung Tumor Progression through Neutrophil Attraction to Tumor Sites and Mediating Resistance to PD-1 Blockade. J Thorac Oncol. 2017; 12(8):1268-79. Epub 20170506. doi: 10.1016/j.jtho.2017.04.017. PubMed PMID: 28483607; PubMed Central PMCID: PMCPMC5532066.
55. Wu L, Awaji M, Saxena S, Varney M L, Sharma B, Singh R K. IL-17-CXC Chemokine Receptor 2 Axis Facilitates Breast Cancer Progression by Up-Regulating Neutrophil Recruitment. Am J Pathol. 2020; 190(1):222-33. Epub 20191022. doi: 10.1016/j.ajpath.2019.09.016. PubMed PMID: 31654638; PubMed Central PMCID: PMCPMC6943375.
56. Stuart W D, Fink-Baldauf I M, Tomoshige K, Guo M, Maeda Y. CRISPRi-mediated functional analysis of NKX2-1-binding sites in the lung. Commun Biol. 2021; 4(1):568. Epub 20210512. doi: 10.1038/s42003-021-02083-4. PubMed PMID: 33980985; PubMed Central PMCID: PMCPMC8115294.
57. Papalexi E, Satija R. Single-cell RNA sequencing to explore immune cell heterogeneity. Nat Rev Immunol. 2018; 18(1):35-45. Epub 20170807. doi: 10.1038/nri.2017.76. PubMed PMID: 28787399.
58. Williams C G, Lee H J, Asatsuma T, Vento-Tormo R, Haque A. An introduction to spatial transcriptomics for biomedical research. Genome Med. 2022; 14(1):68. Epub 20220627. doi: 10.1186/s13073-022-01075-1. PubMed PMID: 35761361; PubMed Central PMCID: PMCPMC9238181.
59. Reid M D, Basturk O, Thirabanjasak D, Hruban R H, Klimstra D S, Bagci P, et al. Tumor-infiltrating neutrophils in pancreatic neoplasia. Mod Pathol. 2011; 24(12):1612-9. Epub 20110805. doi: 10.1038/modpathol.2011.113. PubMed PMID: 21822201; PubMed Central PMCID: PMCPMC3227778.
60. Kuo M H, Lee A C, Hsiao S H, Lin S E, Chiu Y F, Yang L H, et al. Cross-talk between SOX2 and TGFβ Signaling Regulates EGFR-TKI Tolerance and Lung Cancer Dissemination. Cancer Res. 2020; 80(20):4426-38. Epub 20200819. doi: 10.1158/0008-5472.Can-19-3228. PubMed PMID: 32816907.
61. Robinson J T, Thorvaldsdóttir H, Winckler W, Guttman M, Lander E S, Getz G, et al. Integrative genomics viewer. Nat Biotechnol. 2011; 29(1):24-6. doi: 10.1038/nbt.1754. PubMed PMID: 21221095; PubMed Central PMCID: PMCPMC3346182.

What is claimed is:

1. A method for predicting clinical outcomes of a lung cancer patient, comprising providing a cancer cell sample of the patient, and detecting the expression level of NKX2-1 in the sample, wherein the clinical outcome is predicted as poor if the expression of NKX2-1 is low.

2. The method of claim 1, wherein the lung cancer is lung adenocarcinoma (LUAD).

3. A method for treating lung adenocarcinoma (LUAD) in a patient, comprising administering to said patient an inhibitor of CXCR2 at a therapeutically effective amount.

4. The method of claim 3, wherein the LUAD is a cancer with low expression of NKX2-1.

5. The method of claim 3, wherein the inhibitor of CXCR2 is CXCL1, CXCL2, or CXCL5.

6. The method of claim 3, wherein the inhibitor of CXCR2 is a CXCR2 antagonist, which comprises SB225002, MK-7123, Cpd 19, or AZD5069.
